SEQUENCE LISTING (22 sequences)

SEQ ID NO: 1 - DNA, 17 bp, synthetic construct
  source 1..17: mol_type = other DNA
tgaaggtaaccgggata

SEQ ID NO: 2 - DNA, 24 bp, synthetic construct
  source 1..24: mol_type = other DNA
tgaaggtaaccgggatatgcaaga

SEQ ID NO: 3 - DNA, 17 bp, synthetic construct
  source 1..17: mol_type = other DNA
tatcccggttaccttca

SEQ ID NO: 4 - DNA, 24 bp, synthetic construct
  source 1..24: mol_type = other DNA
tcttgcatatcccggttaccttca

SEQ ID NO: 5 - DNA, 24 bp, synthetic construct
  source 1..24: mol_type = other DNA
aagagggtgaaggtaaccgggata

SEQ ID NO: 6 - DNA, 24 bp, synthetic construct
  source 1..24: mol_type = other DNA
cagatgaaagagggtgaaggtaac

SEQ ID NO: 7 - DNA, 24 bp, synthetic construct
  source 1..24: mol_type = other DNA
tatcccggttaccttcaccctctt

SEQ ID NO: 8 - DNA, 24 bp, synthetic construct
  source 1..24: mol_type = other DNA
gttaccttcaccctctttcatctg

SEQ ID NO: 9 - RNA, 24 nt, synthetic construct
  source 1..24: mol_type = other RNA
  modified_base order(2,11,17): mod_base = OTHER; note = internal 2'-O-methoxyethly adenine
  modified_base order(4..6,12..13,16,18..20,22): mod_base = OTHER; note = internal 2'-O-methoxyethly cytosine
  modified_base 7..8: mod_base = OTHER; note = internal 2'-O-methoxyethly guanine
  modified_base order(3,9..10,14..15,21,23): mod_base = OTHER; note = internal 2'-O-methoxyethly thymine
  modified_base 1: mod_base = OTHER; note = 5' 2'-O-methoxyethyl thymine
  modified_base 24: mod_base = OTHER; note = 3' 2'-O-methoxyethyl thymine
  modified_base 1..24: mod_base = OTHER; note = phosphorothioate bond
tatcccggttaccttcaccctctt

SEQ ID NO: 10 - RNA, 24 nt, synthetic construct
  source 1..24: mol_type = other RNA
  modified_base order(4,10,20): mod_base = OTHER; note = internal 2'-O-methoxyethly adenine
  modified_base order(5..6,9,11..13,15,19,22): mod_base = OTHER; note = internal 2'-O-methoxyethly cytosine
  modified_base order(2..3,7..8,14,16..18,21,23): mod_base = OTHER; note = internal 2'-O-methoxyethly thymine
  modified_base 1: mod_base = OTHER; note = 5' 2'-O-methoxyethyl guanine
  modified_base 24: mod_base = OTHER; note = 3' 2'-O-methoxyethyl guanine
  modified_base 1..24: mod_base = OTHER; note = phosphorothioate bond
gttaccttcaccctctttcatctg

SEQ ID NO: 11 - RNA, 24 nt, synthetic construct
  source 1..24: mol_type = other RNA
  modified_base order(7,9,18): mod_base = OTHER; note = internal 2'-O-methoxyethly adenine
  modified_base order(2,6,11..13,19..20,23): mod_base = OTHER; note = internal 2'-O-methoxyethly cytosine
  modified_base order(5,14..15): mod_base = OTHER; note = internal 2'-O-methoxyethly guanine
  modified_base order(3..4,8,10,16..17,21..22): mod_base = OTHER; note = internal 2'-O-methoxyethly thymine
  modified_base 1: mod_base = OTHER; note = 5' 2'-O-methoxyethyl thymine
  modified_base 24: mod_base = OTHER; note = 3' 2'-O-methoxyethyl adenine
  modified_base 1..24: mod_base = OTHER; note = phosphorothioate bond
tcttgcatatcccggttaccttca

SEQ ID NO: 12 - DNA, 24 bp, synthetic construct
  source 1..24: mol_type = other DNA
attttttggcctcttgtttagtgg

SEQ ID NO: 13 - DNA, 24 bp, synthetic construct
  source 1..24: mol_type = other DNA
ggcctcttgtttagtggactgtgg

SEQ ID NO: 14 - DNA, 24 bp, synthetic construct
  source 1..24: mol_type = other DNA
gtctttcttttctgtttttacctt

SEQ ID NO: 15 - DNA, 24 bp, synthetic construct
  source 1..24: mol_type = other DNA
ccactaaacaagaggccaaaaaat

SEQ ID NO: 16 - DNA, 24 bp, synthetic construct
  source 1..24: mol_type = other DNA
ccacagtccactaaacaagaggcc

SEQ ID NO: 17 - DNA, 24 bp, synthetic construct
  source 1..24: mol_type = other DNA
aaggtaaaaacagaaaagaaagac

SEQ ID NO: 18 - RNA, 24 nt, synthetic construct
  source 1..24: mol_type = other RNA
  modified_base order(3,6..8,10..11,13,18..23): mod_base = OTHER; note = internal 2'-O-methoxyethly adenine
  modified_base order(2,4,9,16..17): mod_base = OTHER; note = internal 2'-O-methoxyethly cytosine
  modified_base order(12,14..15): mod_base = OTHER; note = internal 2'-O-methoxyethly guanine
  modified_base 5: mod_base = OTHER; note = internal 2'-O-methoxyethly thymine
  modified_base 1: mod_base = OTHER; note = 5' 2'-O-methoxyethyl cytosine
  modified_base 24: mod_base = OTHER; note = 3' 2'-O-methoxyethyl thymine
  modified_base 1..24: mod_base = OTHER; note = phosphorothioate bond
ccactaaacaagaggccaaaaaat

SEQ ID NO: 19 - RNA, 24 nt, synthetic construct
  source 1..24: mol_type = other RNA
  modified_base order(3,5,10,13..15,17..18,20): mod_base = OTHER; note = internal 2'-O-methoxyethly adenine
  modified_base order(2,4,8..9,11,16,23): mod_base = OTHER; note = internal 2'-O-methoxyethly cytosine
  modified_base order(6,19,21..22): mod_base = OTHER; note = internal 2'-O-methoxyethly guanine
  modified_base order(7,12): mod_base = OTHER; note = internal 2'-O-methoxyethly thymine
  modified_base 1: mod_base = OTHER; note = 5' 2'-O-methoxyethyl cytosine
  modified_base 24: mod_base = OTHER; note = 3' 2'-O-methoxyethyl cytosine
  modified_base 1..24: mod_base = OTHER; note = phosphorothioate bond
ccacagtccactaaacaagaggcc

SEQ ID NO: 20 - RNA, 24 nt, synthetic construct
  source 1..24: mol_type = other RNA
  modified_base order(2,6..10,12,14..17,19..21,23): mod_base = OTHER; note = internal 2'-O-methoxyethly adenine
  modified_base 11: mod_base = OTHER; note = internal 2'-O-methoxyethly cytosine
  modified_base order(3..4,13,18,22): mod_base = OTHER; note = internal 2'-O-methoxyethly guanine
  modified_base 5: mod_base = OTHER; note = internal 2'-O-methoxyethly thymine
  modified_base 1: mod_base = OTHER; note = 5' 2'-O-methoxyethyl adenine
  modified_base 24: mod_base = OTHER; note = 3' 2'-O-methoxyethyl cytosine
  modified_base 1..24: mod_base = OTHER; note = phosphorothioate bond
aaggtaaaaacagaaaagaaagac

SEQ ID NO: 21 - DNA, 59 bp, synthetic construct
  source 1..59: mol_type = other DNA
gcatattttttggcctcttgtttagtggactgtggtgtctttcttttctgtttttactt

SEQ ID NO: 22 - DNA, 53 bp, synthetic construct
  source 1..53: mol_type = other DNA
agagaagaacagatgaaagagggtgaaggtaaccgggatatgcaagagctgca

(19) United States
(12) Patent Application Publication
(10) Pub. No.: US 2026/0062701 A1
(43) Pub. Date: Mar. 5, 2026

(54) COMPOSITIONS AND METHODS FOR TREATING MENINGIOMA

(71) Applicant: The Jackson Laboratory, Bar Harbor, ME (US)

(72) Inventors: Olga Anczuków-Camarda, Bar Harbor, ME (US); Nathan Leclair, Bar Harbor, ME (US)

(73) Assignee: The Jackson Laboratory, Bar Harbor, ME (US)

(21) Appl. No.: 19/304,919

(22) Filed: Aug. 20, 2025

Related U.S. Application Data
(60) Provisional application No. 63/685,365, filed on Aug. 21, 2024.

(51) Int. Cl. C12N 15/113; A61K 31/7115; A61K 31/712; A61K 31/7125

(52) U.S. Cl. CPC C12N 15/113; A61K 31/7115; A61K 31/712; A61K 31/7125; C12N 2310/11; C12N 2310/315; C12N 2310/321; C12N 2310/33; C12N 2310/346; C12N 2320/33

(57) ABSTRACT

The disclosure relates to methods and compositions that regulate splicing in NASP transcripts.

### RELATED APPLICATION

This application claims the benefit under 35 U.S.C. § 119(e) of U.S. provisional application No. 63/685,365 filed Aug. 21, 2024, which is incorporated by reference herein in its entirety.

### GOVERNMENT LICENSE RIGHTS

This invention was made with government support under CA034196, CA262311, NS118039, NS117104, and CA221747 awarded by National Institutes of Health. The government has certain rights in the invention.

### REFERENCE TO AN ELECTRONIC SEQUENCE LISTING

The content of the electronic sequence listing (J022770159US01-SEQ-HJD.xml; Size: 43,953 bytes; and Date of Creation: Aug. 20, 2025) is herein incorporated by reference in its entirety.

### BACKGROUND

Advances in the understanding of the molecular biology of meningiomas have led to significant gains in the ability to predict patient prognosis and tumor recurrence and to identify novel targets for therapeutic design. Specifically, classification of meningiomas based on DNA methylation has improved the ability to risk stratify patients; however, new questions have arisen in terms of the underlying impact these DNA methylation signatures have on meningioma biology.

### SUMMARY

DNA methylation profiling has revealed molecular groups of meningiomas that are associated with distinct gene expression programs, therapeutic vulnerabilities, and clinical outcomes. Nonetheless, RNA processing events across meningioma DNA methylation groups have not been assessed. Specifically, RNA splicing, a key step in gene expression that promotes transcriptomic and proteomic diversity, has yet to be systematically characterized in human meningiomas. The work described in this disclosure identifies key RNA splicing events associated with high-risk (Hypermitotic) meningioma groups. RNA binding proteins that are differentially expressed and that regulate these splicing events in meningiomas were also identified. Finally, splice-switching antisense oligonucleotides directed at oncogenic splicing events that are toxic to Hypermitotic meningioma cell lines in vitro were created. Together, the experiments presented herein provides the first systematic identification of alternative splicing events across molecular groups of meningiomas with potential utility in clinical diagnostics, prognostication, and therapeutics.

More specifically, this body of work utilizes RNA-seq data from 486 meningioma samples corresponding to three meningioma DNA methylation groups (Merlin-intact, Immune-enriched, and Hypermitotic), followed by in vitro experiments utilizing human meningioma cell lines. Alterations in RNA splicing between meningioma DNA methylation groups were identified, including individual splicing events that correlate with Hypermitotic meningiomas and predict tumor recurrence and overall patient prognosis; and a set of splicing events that can accurately predict DNA methylation classification based on RNA-seq data was compiled. Furthermore, these events were validated using RT-PCR in patient samples and meningioma cell lines. Additionally, alterations in RNA binding proteins and splicing factors that lie upstream of RNA splicing events were identified, including upregulation of SRSF1 in Hypermitotic meningiomas, which was shown to drive alternative RNA splicing changes. Finally, splice switching antisense oligonucleotides to target RNA splicing changes in NASP and MFF observed in Hypermitotic meningiomas were designed, providing a rationale for RNA-based therapeutic design. This disclosure provides evidence that RNA splicing is an important driver of meningioma phenotypes that can be useful in prognosticating patients and as a potential exploit for therapeutic vulnerabilities.

Some aspects relate to an engineered splice-switching antisense oligonucleotide (SSO) that binds to the nucleotide sequence of SEQ ID NO: 1. In some embodiments, the engineered splice-switching antisense oligonucleotide binds to the nucleotide sequence of SEQ ID NO: 2.

Other aspects relate to an engineered splice-switching antisense oligonucleotide comprising the nucleotide sequence of SEQ ID NO: 3. In some embodiments, the engineered splice-switching antisense oligonucleotide comprises the nucleotide sequence of SEQ ID NO: 4 or a nucleotide sequence having at least 90% identity to the nucleotide sequence of SEQ ID NO: 4. In some embodiments, the engineered splice-switching antisense oligonucleotide of comprises a nucleotide sequence having at least 95% identity to the nucleotide sequence of SEQ ID NO: 4. In some embodiments, the engineered splice-switching antisense oligonucleotide comprises the nucleotide sequence of SEQ ID NO: 4.

In some embodiments, an engineered splice-switching antisense oligonucleotide comprises a chemical modification. In some embodiments, a chemical modification is selected from backbone modifications, sugar modifications, and base modifications. In some embodiments, a chemical modification is a backbone modification. In some embodiments, a backbone modification is a phosphorothioate (PS) modification. In some embodiments, a chemical modification is a sugar modification. In some embodiments, a sugar modification is 2′-O-Methyl (2′-OMe) or 2′-O-Methoxyethyl (2′-MOE).

Some aspects relate to a composition comprising an engineered splice-switching antisense oligonucleotide described herein and an excipient.

Other aspects relate to a composition comprising an engineered splice-switching antisense oligonucleotide described herein and a delivery vehicle. In some embodiments, a delivery vehicle is selected from lipid nanoparticles, liposomes, polymeric nanoparticles, gold nanoparticles, peptide-based delivery systems, aptamer-based delivery systems, exosomes, viral vectors, and molecules capable of crossing the blood-brain barrier.

In some embodiments, the concentration of an engineered splice-switching antisense oligonucleotide in the composition is about 2 mg/ml to about 200 mg/ml.

Further aspects relate to a method comprising administering an engineered splice-switching antisense oligonucleotide described herein, or a composition described herein, to a subject, for example, a subject having (e.g., diagnosed as having) a meningioma.

In some embodiments, an engineered splice-switching antisense oligonucleotide is administered via intrathecal injection, intracranial injection, or intravenous infusion.

In some embodiments, a meningioma is classified as Hypermitotic meningioma.

In some embodiments, an engineered splice-switching antisense oligonucleotide or a composition is administered in an amount effective to cause meningioma cell death.

Still other aspects relate to a method comprising administering an engineered splice-switching antisense oligonucleotide described herein to a cell (or contacting a cell with an engineered SSO), for example, a meningioma cell, in an amount effective to modify splicing of NASP transcript. In some embodiments, a cell is a brain cell.

### BRIEF DESCRIPTION OF THE DRAWINGS

FIGS. 1A-1R. RNA splicing differences underlie meningioma DNA methylation groups: (FIG. 1A) Description of human meningioma samples (n=486), split into discovery (n=184) and validation (n=302) cohorts. Meningioma samples were subjected to DNA methylation profile and group assignment, as described previously, as well as RNA-sequencing. Histograms represent the relative proportion of meningiomas in each cohort by DNA methylation group, conventional WHO grade, and recurrence status. (FIG. 1B) RNA-sequencing data was subjected to a computational pipeline that predicts five types of alternative RNA splicing events (CA: Cassette Alternative exon; MXE: Mutually Exclusive Exon, A3SS/A5SS: Alternative 3′/5′ Splice Site, RI: Retained Intron) based on exonic, intronic and junctional read counts. (FIG. 1C) Summary plots displaying significant differential AS events (ΔPercent Spliced In (PSI)>10%, FDR<0.05, p<0.05) between DNA methylation groups in the discovery cohort. Pie charts demonstrate distribution of AS events by event type (CA: Cassette Alternative exon; MXE: Mutually Exclusive Exon, A3SS/A5SS: Alternative 3′/5′ Splice Site, RI: Retained Intron), histograms depict total count of AS events by ΔPSI. (FIG. 1D) Overlap of significant AS events detected across DNA methylation group comparisons. (FIG. 1E) Significant AS events detected in the discovery cohort are plotted as a heatmap of PSI z-score across tumor samples. Hierarchical clustering based on Euclidean distance. (FIG. 1F) Proportion of tumors representative of each DNA methylation group in the hypermitotic splicing signature cluster from (FIG. 1E) vs the remainder of meningioma samples in the discovery cohort. (FIGS. 1G-1J) NASP-CA inclusion was quantified for both the discovery (circle points) and validation cohorts (triangle points) and displayed as PSI across WHO grades (FIG. 1E), DNA methylation groups (FIG. 1F), and recurrence status (FIG. 1G) (median±IQR, Wilcoxon test; *p<0.05, **p<0.01, ***p<0.001, ****p<0.0001, ns—not significant). Tumors were stratified based on NASP-CA inclusion level as high (z-score>0.5), low (z-score<−0.5), or other (−0.5<z-score>0.5). Patient outcomes were assessed with Kaplan-Meier estimates of local freedom from recurrence (LFFR) (left) and overall survival (OS) (right) (FIG. 1H). (FIGS. 1K-1N) hnRNPM-RI inclusion across cohorts (FIGS. 1K-1M), and across patient outcomes (FIG. 1N), same as in (FIGS. 1G-1J). (FIGS. 10-1R) MFF-CA inclusion across cohorts (FIGS. 10-1Q), and across patient outcomes (FIG. 1R), same as in (FIGS. 1G-1J).

FIGS. 2A-2B. Alternative RNA splicing patterns accurately predict meningioma DNA methylation groups: (FIG. 2A) PSI values from 74 AS events that were significantly different between DNA methylation groups in the discovery cohort and identified in the validation cohort are plotted as z-score of mean group PSI across both cohorts. Hierarchical clustering by Euclidean distance. (FIG. 2B) Z-scaled PSI values (FIG. 2A) were used as input variables for a random forest algorithm trained on the discovery cohort and tested on the validation cohort. Receiver operating curves for this random forest classifier were applied to the validation cohort. Area under the curve (AUC) values are shown in the bar plots (AUC±95% CI)

FIGS. 3A-3H. Classification of meningioma cell lines and patient samples using PCR-based detection of alternative RNA splicing events: (FIG. 3A) Cell lines derived from human meningioma tumors previously described to have DNA-methylation profiles consistent with merlin-intact (UCSF-HO, UCSF-ID), immune-enriched (NU02141, NU02171, IOMM-Lee), and hypermitotic meningiomas (BenMen). (FIGS. 3B-3D) Splicing of NASP-CA (FIG. 3F), MFF-CA (FIG. 3G), and hnRNPM-RI (FIG. 3H) was measured across meningioma cell lines using RT-PCR primers that amplify both included and skipped isoforms. Representative gels along with percent spliced (PSI) quantification from band intensity are shown (n=3; mean±SD; ANOVA). (FIG. 3E) Axial post-gadolinium contrast T1-weighted MRI sequences from a patient with right orbital meningioma demonstrating the original tumor and recurrence. (FIGS. 3F-3H) Splicing of NASP-CA (FIG. 3F), MFF-CA (FIG. 3G), or hnRNPM-RI (FIG. 3H) was measured in primary and recurrent tumor samples from (FIG. 3E) using RT-PCR primers that amplify both included and skipped isoforms. Representative gels along with percent spliced (PSI) quantification from band intensity are shown (ΔPSI=PSI_{recurrent}−PSI_{primary}).

FIGS. 4A-4C. Splicing factor expression is dysregulated across meningioma DNA methylation groups: (FIG. 4A) Gene expression of 770 RBPs across meningioma DNA methylation groups using DESeq2. Normalized counts were extracted and z-scaled by cohort (discovery vs. validation) and plotted as a heatmap with hierarchical clustering based on Euclidean distance (top). Metadata provided on the right highlights each RBPs role in known RNA processing steps. Gene expression data from both cohorts was then aggregated and differential expression of RBPs was further compared between DNA-methylation groups and represented as Log₂FC (bottom, plots scaled to Log₂FC of ±1) with downregulated genes in blue (Log₂FC<0, p_{adj}<0.05) and upregulated genes in red (Log₂FC>0, p_{adj}<0.05). (FIG. 4B) Expression of RBPs with enrichment in Merlin-intact (RBFOX2, SRSF12), Immune-enriched (SNRNP40, MBNL1), and Hypermitotic meningiomas (LARP1, SRSF1, LUC7L). Z-scaled normalized counts of each respective RBP across tumor samples is plotted by DNA methylation group (n=486; median±IQR; Wilcoxon test, *p<0.05, **p<0.01, ***p<0.001, ****p<0.0001, ns—not significant).

FIGS. 5A-5P. SRSF1 is upregulated in hypermitotic meningiomas and contributes to splicing changes and meningioma cell proliferation in vitro: (FIG. 5A) SRSF1 gene expression was quantified from RNA-seq across both discovery and validation cohorts. Z-scaled normalized counts across tumor samples are plotted for each DNA methylation group (n=486; median±IQR; Wilcoxon test; *p<0.05, **p<0.01, ***p<0.001, ****p<0.0001, ns—not significant). (FIG. 5B) Relationship of SRSF1 expression on patient outcomes was assessed by first stratifying patients based on SRSF1 expression (top) or first by meningioma DNA-methylation and then by SRSF1 expression (bottom) as high (z-score>0.5) or low (z-score<0.5). Survival outcomes were assessed with Kaplan-Meier estimates of overall survival (OS) (left) and local freedom from recurrence (LFFR) (right). (FIG. 5C) SRSF1 protein expression was assessed across meningioma cell lines, representative of different DNA-methylation groups, by western blotting with antibodies directed against SRSF1 and normalized to total protein input. Representative gels along with quantification are shown (n=3, mean±SD, ANOVA). (FIGS. 5D, 5I) Expression of SRSF1 in BenMen (FIG. 5D) and IOMM-Lee (FIG. 5I) cells transfected with siRNAs targeting SRSF1 or a negative control assessed at 48 h post-transfection using western blotting and normalized to GAPDH as a loading control. Representative gels along with quantification are shown (n=3; mean±SD; t-test to control, **p<0.01, ****p<0.0001 ns—not significant). (FIGS. 5E, 5J) Cell proliferation in BenMen (FIG. 5E) and IOMM-Lee (FIG. 5J) cells transfected with siRNAs targeting SRSF1 or a negative control assessed using an EdU incorporation assay, counterstained with Hoechst, and plotted as percent EdU+ to total Hoechst+ cells (n=3; mean±SD; t-test to control, *p<0.05, **p<0.01, ****p<0.0001 ns—not significant). (FIGS. 5F, 5M) Splicing of NASP-CA (FIGS. 5F, 5K), MFF-CA (FIGS. 5G, 5L), and hnRNPM-RI (FIGS. 5H, 5M) in BenMen (FIGS. 5F, 5G, 5H) and IOMM-Lee (FIGS. 5K, 5L, 5M) cells transfected with siRNAs targeting SRSF1 or a negative control assessed with RT-PCR primers that amplify both included and skipping bands. Representative gels along with PSI quantification from band intensity are shown (n=3; mean±SD; t-test to control, *p<0.05, **p<0.01, ***p<0.001, ****p<0.0001, ns—not significant). (FIGS. 5N-5P) Location of SRSF1 eCLIP peaks from ENCODE HepG2 and K562 cell lines in exonic and intronic regions nearby the NASP-CA (FIG. 5N), MFF-CA (FIG. 50), and hnRNPM-RI (FIG. 5P) AS events. Individual boxes represent peak calls for each replicate (R1 and R2) for indicated cell lines. Upper diagram shows the genomic region, middle diagram shows the transcript structure of the specific AS event.

FIGS. 6A-6K. Splice switching ASO targeting the hypermitotic meningioma associated NASP-CA splicing event are toxic to meningioma cells in vitro: (FIG. 6A) ASO-NASP #3 targets the 5′ splice site (5′SS) of NASP-CA resulting in increased exon skipping. Schematic structure of the entire NASP gene is shown on top, along with the zoom-in region with the NASP-CA exon in green and the relative position of ASO-NASP #3. The predicted splicing consequences are shown on the right. (FIGS. 6B, 6F) NASP-CA splicing in BenMen (FIG. 6B) and IOMM-Lee (FIG. 6F) cells transfected with 200 nM of ASO-NASP #3 or ASO-CTL is measured 48 h post-transfection by RT-PCR using primers that amplify the skipped and included isoform. Representative gels along with percent spliced (PSI) quantification from band intensity are shown (n=3, mean±SD, t-test; ****p<0.0001). (FIGS. 6C, 6G) NASP protein isoform expression in BenMen (FIG. 6C) and IOMM-Lee (FIG. 6G) cells transfected with 200 nM of ASO-NASP #3 or ASO-CTL is measured 48 h post-transfection by western blotting using an antibody that recognizes both the high molecular weight (H-MW; tNASP) and low molecular weight (L-MW; sNASP) isoforms, or GAPDH for loading control. Protein isoforms are normalized to GAPDH and then represented as a percentage of total NASP protein. Representative gels along with quantification are shown (n=3, mean±SD, t-test; *p<0.05, ns—not significant). (FIGS. 6D, 6H) Cell death in BenMen (FIG. 6D) and IOMM-Lee (FIG. 6H) cells transfected with 200 nM of ASO-NASP #3 or ASO-CTL is measured 48 h post-transfection with a live stain caspase-3/7 activation detection reagent (cell event), counterstained with Hoechst and propidium iodide (PI), and plotted as percent caspase+ to total Hoechst+ cells. Representative images (scale bar: X um) along with quantification are shown (n=3, mean±SD, t-test; ***p<0.001). (FIGS. 6E, 6I) Cell proliferation in BenMen (FIG. 6E) and IOMM-Lee (FIG. 6I) cells transfected with 200 nM of ASO-NASP #3 or ASO-CTL is measured 48 h post-transfection using an EdU incorporation assay, counterstained with Hoechst, and plotted as percent EdU+ to total Hoechst+ cells. Representative images (scale bar: 200 um) along with quantification are shown (n=3, mean±SD, t-test; *p<0.05, ****p<0.0001). (FIGS. 6J, 6K) NASP protein localization in BenMen (FIG. 6J) and IOMM-Lee (FIG. 6K) cells transfected with 200 nM of ASO-NASP #3 or ASO-CTL is measured 48 h post-transfection using immunofluorescence and counterstained with Hoechst and phalloidin (F-actin). Relative nuclear to cytoplasmic distribution of NASP is quantified as the total nuclear to total cytoplasmic NASP fluorescence. Representative images (scale bar: 50 um) along with quantification are shown (n=8 individual fields; mean±SD; t—test; ns—not significant, *p<0.05, **p<0.01, ***p<0.001, ****p<0.0001).

FIGS. 7A-7G. Splicing modulation of Hypermitotic meningioma related AS events in NASP and MFF using RNA-targeting splice-switching ASO: (FIG. 7A) Position of three 24mer ASOs designed to target an intronic splicing silencer (ISS) and hnRNPA1 silencer site downstream of the MFF-CA event detected in hypermitotic meningiomas (SEQ ID NO: 21). (FIG. 7B) MFF-CA splicing in BenMen and IOMM-Lee cells transfected with 200 nM control or MFF-ISS targeting ASOs is assessed at 48 h post-transfection using RT-PCR with primers that amplify both the included and skipped isoforms. PSI was quantified from band intensity. (FIG. 7C) MFF-CA splicing in BenMen cells transfected with increasing concentrations (50 nM, 100 nM, 200 nM, 500 nM) of control or MFF-ISS ASOs is assessed at 48 h post-transfection using RT-PCR with primers that amplify both the included and skipped isoforms. PSI was quantified from band intensity (n=3; mean±SD; t-test to control, ***p<0.001, ****p<0.0001). (FIG. 7D) Position of three 24mer ASOs designed to target the 5′SS in the NASP-CA event detected in hypermitotic meningiomas. Each ASO has variable overlap with the 5′SS (SEQ ID NO: 22). (FIG. 7E) NASP-CA splicing in BenMen and IOMM-Lee cells transfected with 200 nM control or NASP-5SS targeting ASOs is assessed at 48 h post-transfection using RT-PCR with primers that amplify both the included and skipped isoforms. PSI was quantified from band intensity. (FIG. 7F) NASP-CA splicing in BenMen cells transfected with increasing concentrations (50 nM, 100 nM, 200 nM, 500 nM) of control or NASP-5SS ASOs is assessed at 48 h post-transfection using RT-PCR with primers that amplify both the included and skipped isoforms. PSI was quantified from band intensity (n=3; mean±SD; t-test to control, ***p<0.001, ****p<0.0001). (FIG. 7G) Cell viability in BenMen cells transfected with 200 nM of ASOs targeting NASP-5SS, MFF-ISS, or control is assessed at 48 h post-transfection using a cell titer glow assay (n=3; mean±SD; t-test to control, *p<0.05, **p<0.01, ***p<0.001).

### DETAILED DESCRIPTION

Meningiomas are the most common intracranial tumor. While most are benign, a subset is aggressive with high rates of recurrence despite standard treatment. Recent studies have revised meningioma classifications based on tumor genomics, epigenetics, and gene expression signatures, and nominated novel therapeutic targets that are in pre-clinical or early clinical trials for aggressive meningiomas. Among these molecular approaches to meningioma classification, DNA methylation is a powerful tool for predicting patient outcomes. However, the field lacks a comprehensive understanding of the biological differences between DNA methylation groups and the downstream impact of these changes on tumor growth or therapeutic vulnerability. Interestingly, DNA methylation groups with more malignant tumors harbor dysregulated expression signatures across genes involved in RNA processing and splicing. Alternative RNA splicing (AS) is a key step in gene expression regulation that allows individual genes to encode multiple RNA isoforms, facilitating transcriptomic diversity that underlies cellular phenotypes. AS is dysregulated in cancers where the expression of RNA isoforms is skewed towards those that promote hallmark phenotypes of cancer. This shift in RNA isoforms is due to underlying defects in RNA processing machinery including splicing factors (SFs), a family of RNA binding proteins (RBPs) that regulate AS in a dose-dependent manner. SFs are recurrently mutated in hematological malignancies but predominantly undergo copy number and expression level changes in solid tumors. Despite its importance in cancer biology, a systematic analysis of AS changes in meningiomas is lacking. A few studies examined the impact of individual AS isoforms on meningioma tumorigenesis, revealing important interactions with isoforms of CHEK2 and NF2 loss of function, alternative splicing in NF2 itself, and tumor dependency on RBPs. These findings suggest a role for AS in meningioma biology, and an unbiased high-throughput analysis might therefore discover key RNA isoforms that impact meningioma tumorigenesis.

As described herein, 486 meningioma samples were systematically analyzed, revealing differences in AS patterns across DNA methylation groups, identifying AS events that predict tumor recurrence and overall survival across independent patient cohorts. Additionally, the work described herein resulted in the identification of upstream RBPs that are differentially expressed between DNA methylation groups, including those upregulated in Hypermitotic meningiomas, which have the worst clinical outcomes. This body of work also demonstrated that depletion of these proteins impairs meningioma cell proliferation. Finally, splice switching antisense oligonucleotides (ASOs) that target Hypermitotic-associated AS events in NASP and MFF were develop, providing a proof-of-principle in rational therapeutic design against AS in meningiomas.

### Alternative RNA Splicing (AS)

Alternative RNA splicing is a regulated process during gene expression that allows a single gene to produce multiple protein isoforms. There are several types of alternative splicing, including, for example, exon skipping (an exon may be included or excluded from the final mRNA), mutually exclusive exons (two or more exons are spliced in a way that only one of them is included in the mature mRNA, and intron retention (an intron that is normally spliced out can be retained in the mature mRNA), among others.

Dysregulation of AS is a critical step in tumorigenesis observed across all solid tumor types. The study described herein is the first to systematically identify differential AS events in meningiomas, particularly comparing DNA methylation groups with distinct biological behaviors and clinical outcomes. In total, 184 differential AS events across meningioma DNA methylation groups were uncovered and several that scale with clinical outcomes were identify. Together, these AS events provide novel opportunities for diagnostics, patient stratification, and targeted therapies.

Clinical genomics have revolutionized the approach to central nervous system tumors and have been readily adapted into clinical care. With advances in RNA-sequencing, stratification of meningioma patients based on underlying gene expression and now differential splicing has provided additional information to predict tumor recurrence and therapeutic response. However, these approaches come with financial burden and require significant time commitments for tissue preparation and downstream data analysis. PCR-based testing is a fast and inexpensive assay routinely employed in medical diagnostics. AS provides a unique opportunity, being internally normalized, to identify tumors with ‘high-risk’ isoform expression (e.g., NASP-CA, MFF-CA, HNRNPM-RI). Importantly, AS changes are readily detectable by standard PCR methods in patient samples and cell lines and may aid diagnostics in resource-limited settings or non-academic centers. Furthermore, analysis of gene expression or AS events in combination with clinical genomics and DNA methylation profiling allows for enhanced discretion in meningioma patient prognosis.

RNA binding proteins (RBPs) are a large family of proteins with multifunctional roles in splicing, transcription regulation, mRNA localization, mRNA stability and degradation, epitranscriptomics, and translation. Indeed, some of the most Hypermitotic-enriched RNA binding proteins uncovered here not only impact RNA splicing but also regulate other aspects of RNA biology such as mRNA stability and degradation, transport, translation (e.g., DDX39A, MEX3A, IGF2BP1). SRSF1 is a well described splicing factor that regulates splicing in many different human tumors, promoting isoforms that induce cellular transformation. SRSF1 upregulation was identified in Hypermitotic meningiomas where high expression correlates with lower rates of overall survival and local freedom from recurrence. Further, there is evidence that SRSF1 directly interacts with and regulates AS events associated with Hypermitotic meningiomas. Overall, this implicates SRSF1 as both a prognostic and therapeutic target for aggressive meningiomas, however the overall transcriptome changes observed are likely due to a combination of alterations in multiple RBPs/SFs.

Dysregulated AS can be exploited, in some instances, as a therapeutic option for human tumors, including aggressive meningiomas for which there is a paucity of reliable therapies. Herein, we designed ASOs targeting two AS events enriched in Hypermitotic meningiomas, NASP-CA and MFF-CA. This approach reverses these AS events towards patterns seen in more benign meningiomas (Merlin-intact and Immune-enriched) and exhibit significant toxicity to Hypermitotic meningioma cell lines in vitro. Potential synergism with radiation or other therapies warrants additional investigation. For example, given its importance as a histone shuttler, NASP-targeting ASOs could be combined with histone deactylase inhibitors currently tested in aggressive meningiomas (e.g., AR-42). Additionally, mTOR inhibitors (e.g., everolimus) are being explored in clinical trials for meningiomas refractory to conventional treatment, particularly as NF2 mutated tumors upregulate mTOR signaling. Given the relation between mTOR, mitochondrial dynamics, and autophagy, MFF-targeting ASOs may synergize with mTOR inhibitors. Together, the studies described herein provide an initial look at transcriptome diversity, and highlight the prognostic and therapeutic value of AS for meningiomas.

### Splice-Switching Antisense Oligonucleotides

Provided herein, in some aspects, are therapeutic molecules that can be used to modulate splicing patterns. One such example is splice-switching antisense oligonucleotides (SSOs). SSOs include engineered (e.g., synthetic) oligonucleotides that modulate pre-mRNA splicing patterns, for example, to restore or modify gene function. By binding to specific sequences in the pre-mRNA, SSOs can influence the spliceosome's activity, leading to the inclusion or exclusion of particular exons, retention of introns, or the use of alternative splice sites. This targeted approach allows for the correction of splicing defects or the modulation of gene expression to produce therapeutically beneficial isoforms. The design of SSOs, in some embodiments, includes modifications to enhance their stability, binding affinity, and/or resistance to nucleases, for effective delivery and prolonged action within the body.

The typical length of SSOs can vary but, in some embodiments, is within the range of 15 to 35 nucleotides. For example, an SSO can have a length of 15 to 35, 15 to 25, 15 to 20, 20 to 35, 20 to 30, or 25 to 30 nucleotides. In some embodiments, the length of an SSO is no longer than 30 nucleotides. In some embodiments, the length of an SSO is no longer than 25 nucleotides. In some embodiments, the length of an SSO is no longer than 20 nucleotides. In some embodiments, the length of an SSO is at least 10 nucleotides. In some embodiments, the length of an SSO is at least 15 nucleotides. In some embodiments, the length of an SSO is at least 20 nucleotides. In some embodiments, this length of 15 to 35 nucleotides is optimal for achieving specific binding to a target pre-mRNA sequence while, for example, maintaining efficient cellular uptake and biological activity. The precise length of an SSO can be designed to match the target splice site region closely (e.g., 90% to 100%, 95% to 100%, 96%, 97%, 98%, 99%, or 100% identity), to help promote effective modulation of splicing with minimal side effects.

In some embodiments, a splice-switching antisense oligonucleotide binds to the nucleotide sequence of SEQ ID NO: 1 (TGAAGGTAACCGGGATA). In some embodiments, a splice-switching antisense oligonucleotide binds to the nucleotide sequence of SEQ ID NO: 2 (TGAAGGTAACCGGGATATGCAAGA). In some embodiments, a splice-switching antisense oligonucleotide comprises the nucleotide sequence of SEQ ID NO: 3 (TATCCCGGTTACCTTCA). In some embodiments, a splice-switching antisense oligonucleotide comprises a nucleotide sequence having at least 90% identity to the nucleotide sequence of (TCTTGCATATCCCGGTTACCTTCA) SEQ ID NO: 4. In some embodiments, a splice-switching antisense oligonucleotide comprises a nucleotide sequence having at least 95% identity to the nucleotide sequence of SEQ ID NO: 4. In some embodiments, a splice-switching antisense oligonucleotide comprises the nucleotide sequence of SEQ ID NO: 4.

SSOs, in some embodiments, comprise a (one or more) chemical modification, which can enhance stability, binding affinity, specificity, and/or overall therapeutic potential of an SSO. Non-limiting examples of chemical modifications to an SSO include backbone modifications (e.g., phosphorothioate (PS) modifications, phosphoramidate modifications, phosphonate modifications, Peptide Nucleic Acids (PNAs), morpholinos, etc.), sugar modifications (e.g., 2′-O-Methyl (2′-OMe), 2′-O-Methoxyethyl (2′-MOE), Locked Nucleic Acid (LNA), 2′-Fluoro (2′-F) etc.), base modifications (e.g., 5-Methylcytosine (5-MeC), etc.). In some embodiments, an SSO comprises a backbone modification. In some embodiments, an SSO comprises a sugar modification. In some embodiments, an SSO comprises a base modification. comprises a 2′-Ome. In some embodiments, an SSO comprises a base modification. comprises a 2′-MOE. In some embodiments, an SSO is conjugated to a cell-penetrating peptide to facilitate cellular uptake. In some embodiments, an SSO is conjugated to cholesterol or another lipophilic molecule to improve cell membrane permeability.

In some embodiments, a SSO is associated with (e.g., formulated with, encapsulated in, linked to, encoded by, etc.) a delivery vehicle. A delivery vehicle, in the context of nucleic acids for example, is a system or carrier designed to transport nucleic acids, such as SSOs, into target cells or tissues to achieve a therapeutic effect. Effective delivery vehicles protect nucleic acids from degradation, enhance cellular uptake, ensure efficient release at the target site, and/or minimize off-target effects, in some embodiments. Non-limiting examples of delivery vehicles includes lipid nanoparticles, liposomes, polymeric nanoparticles, gold nanoparticles, peptide-based delivery systems, aptamer-based delivery systems, exosomes, viral vectors, and molecules capable of crossing the blood-brain barrier. In some embodiments, a composition comprises an SSO and an LNP. LNPs are widely used, encapsulating nucleic acids in lipid bilayers to protect them from degradation and facilitate cellular uptake. In some embodiments, a composition comprises an SSO and a polymeric nanoparticle. Polymeric nanoparticles, typically made from biocompatible materials such as PLGA, provide controlled release and/or stability. In some embodiments, a composition comprises a viral vector encoding an SSO. Non-limiting examples of viral vectors include adeno-associated viruses (AAVs) and lentiviruses. In some embodiments, a composition comprises an SSO and an exosome, which is a naturally occurring extracellular vesicles that can be engineered for targeted delivery, for example. In some embodiments, a composition comprises an SSO and a cell-penetrating peptide, or the SSO is modified to include a cell-penetrating peptide, to enhance membrane transport. In some embodiments, a composition comprises an SSO and a cationic polymers, such as polyethylenimine (PEI), or a dendrimer. In some embodiments, a composition comprises an SSO and a gold nanoparticle.

### Pharmaceutical Compositions

The SSOs of the disclosure, in some embodiments, are included in (e.g., formulated in) a composition, such as a pharmaceutic composition. Such compositions can include a (one or more) excipient. An excipient is a substance formulated alongside an active ingredient, such as an SSO of the disclosure, serving various roles, for example, to aid in the manufacturing process, enhance stability, improve bioavailability, and/or ensure the safety and/or efficacy of a drug (e.g., SSO) product. Excipients are generally inactive, meaning they do not exert therapeutic effects, but they can be important, in some instances, for the overall performance of the pharmaceutical product. Non-limiting examples of excipients that can be used in the compositions herein include binders, fillers (diluents), disintegrants, lubricants, preservatives, coatings, solvents, stabilizers, and coloring agents. In some embodiments, an excipient is a buffer, such as saline. In some embodiments, a composition is a solution comprising an SSO and a buffer, such as saline.

The concentration of an SSO in any given composition can vary depending at least in part on the intended use and/or excipients in the composition. For example, the concentration of an SSO can range from about 2 mg/ml to about 200 mg/ml. In some embodiments, the concentration of an SSO is about 2 mg/ml to about 100 mg/ml, about 2 mg/ml to about 50 mg/ml, about 50 mg/ml to about 200 mg/ml, or about 50 mg/ml to about 100 mg/ml. In some embodiments, the concentration of an SSO is at least 2 mg/ml, at least 10 mg/ml, or at least 50 mg/ml.

### Methods of Use

The SSOs of the disclosure, in some embodiments, are administered to a subject, such as a human subject. In some embodiments, the subject has a meningioma. A meningioma is a type of tumor that arises from the meninges, which are the protective membranes that cover the brain and spinal cord. Meningiomas are generally slow-growing and are often benign (non-cancerous), but they can be atypical or malignant (cancerous). These tumors are the most common type of primary brain tumors, accounting for about 30% of all brain tumors. Meningiomas are classified by the World Health Organization (WHO) into three grades based on their histological features: Grade I (Benign), which are the most common and least aggressive, with a low risk of recurrence; Grade II (Atypical), which have a higher likelihood of recurrence and may grow more quickly than Grade I meningiomas; and Grade III (Anaplastic/Malignant), which are the most aggressive, with a high rate of recurrence and potential to metastasize. In some embodiments, a subject has a Grade I meningioma. In some embodiments, a subject has a Grade II meningioma. In some embodiments, a subject has a Grade III meningioma.

Based on DNA methylation patterns, meningiomas can be classified into three molecular subgroups: Merlin-intact, immune-enriched, and hypermitotic. These classifications provide insight into the tumor's biological behavior, prognosis, and potential therapeutic approaches.

Merlin-Intact meningiomas retain the function of the Merlin protein, encoded by the NF2 gene, which plays a critical role in regulating cell growth and maintaining cell-cell adhesion. These tumors have a distinct DNA methylation profile/signature, often have an intact NF2 locus, and have the most benign clinical course. Typically corresponding to lower-grade (WHO Grade I) meningiomas, Merlin-intact tumors tend to grow slowly and are less likely to recur after surgical resection. Consequently, patients with Merlin-intact meningiomas generally have a favorable prognosis, with better outcomes and lower rates of recurrence and progression. In some embodiments, a subject has a Merlin-intact meningioma.

Immune-enriched meningiomas are characterized by significant immune cell infiltration and specific DNA methylation patterns associated with immune-related genes. This subgroup features a prominent presence of immune cells such as lymphocytes and macrophages within the tumor microenvironment, which can influence the tumor's behavior and response to therapy. Clinically, immune-enriched meningiomas can vary in presentation and may correspond to both WHO Grade I and Grade II tumors. The immune landscape suggests potential responsiveness to immunotherapeutic approaches, and while prognosis can be variable, some patients may benefit from the body's immune response against the tumor. In some embodiments, a subject has an immune-enriched meningioma.

Hypermitotic meningiomas exhibit high mitotic activity and aggressive growth patterns, with DNA methylation changes indicative of rapid cell division and proliferation. These tumors are typically more aggressive and prone to recurrence, often corresponding to higher-grade tumors (WHO Grade II and Grade III), including atypical and anaplastic meningiomas. The hypermitotic subgroup is marked by methylation patterns linked to genes that regulate the cell cycle and division. Patients with hypermitotic meningiomas generally face a poorer prognosis due to the aggressive nature of these tumors, necessitating more intensive treatment and close monitoring to manage the higher risk of recurrence and progression. In some embodiments, a subject has a hypermitotic meningioma.

Administration of an SSO or a composition comprising an SSO can vary. In some embodiments, an intrathecal route of administration is used. In some embodiments, an intravenous (e.g., infusion or bolus) route of administration is used. In some embodiments, an intracranial route of administration is used.

In some embodiments, an SSO or a composition comprising an SSO is administered in an amount effective to cause or result in cell death. Characteristics of cell death include, for example: morphological changes such as cell shrinkage, chromatin condensation, nuclear fragmentation, cell swelling, loss of membrane integrity, organelle breakdown, and release of cellular contents into the extracellular space; biochemical changes, such as activation of caspases (a family of proteolytic enzymes), externalization of phosphatidylserine on the cell membrane, DNA fragmentation, ATP depletion, uncontrolled release of lysosomal enzymes, and generation of reactive oxygen species (ROS).

In some embodiments, an SSO or a composition comprising an SSO is administered in an amount effective to modify splicing of NASP transcript. NASP (Nuclear Autoantigenic Sperm Protein) is a gene that encodes a protein involved in cell proliferation and chromatin assembly. Splice-switching antisense oligonucleotides targeting NASP, as provided herein, aim to modulate its pre-mRNA splicing to produce specific isoforms of the protein. The two major isoforms are somatic NASP (sNASP) and testicular NASP (tNASP), which differ in their expression patterns and functional roles. SSOs targeting NASP have been designed as described herein to shift the splicing from one isoform to another, resulting in cell death, in some instances. In some embodiments, the cell is a meningioma cell. In some embodiments, the cell is a brain cell.

### EXAMPLES

Leclair N K et al. “RNA splicing as a biomarker and phenotypic driver of meningioma DNA methylation groups,” Neuro-Oncology, noae150, doi.org/10.1093/neuonc/noae150, published 2 Aug. 2024 is incorporated herein by reference in its entirety.

### Example 1

### Alternative RNA Splicing Patterns Distinguish Meningioma DNA Methylation Groups and Predict Patient Outcomes

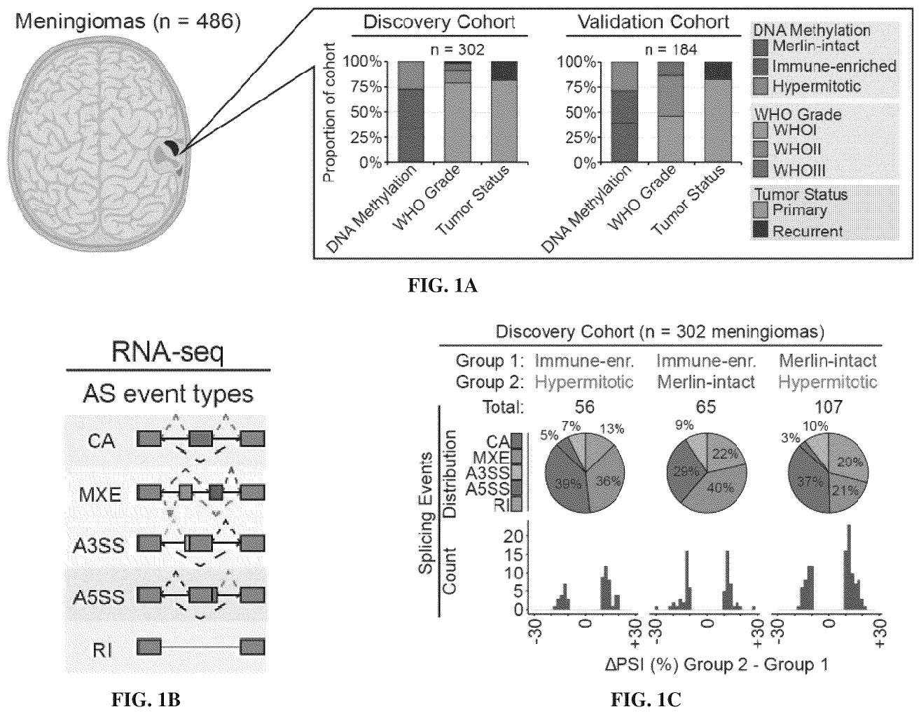

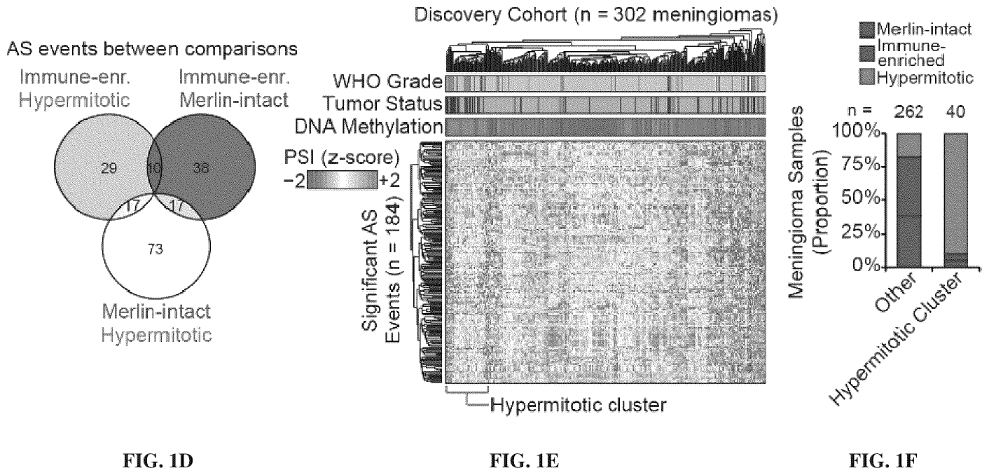

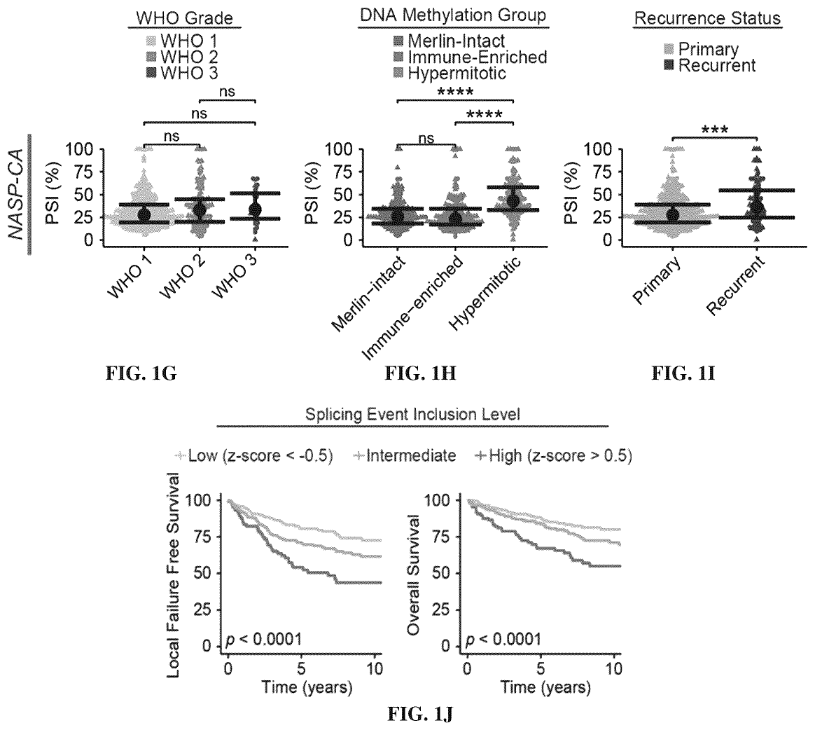

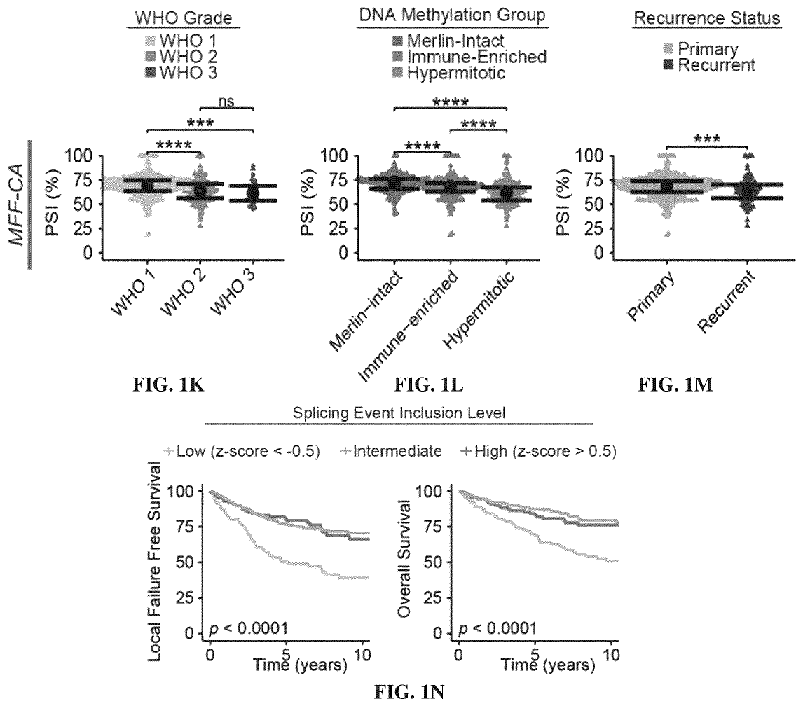

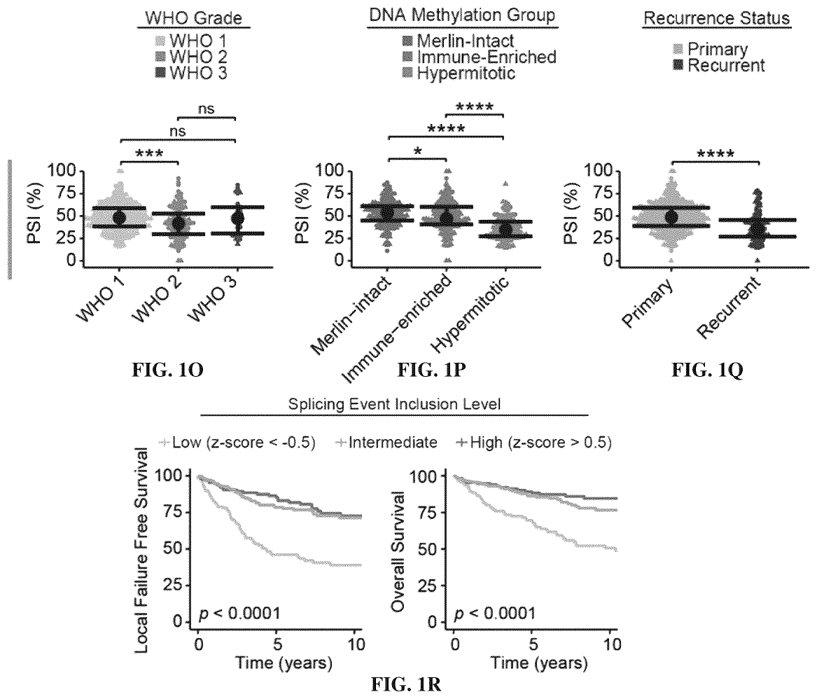

To understand AS differences between meningiomas we analyzed RNA-sequencing data from 486 meningiomas for which there was paired DNA-methylation classification based on the UCSF classifier (Merlin-intact n=176, Immune-enriched n=174, and Hypermitotic n=136), split into discovery (n=302) and validation cohorts (n=184) (FIG. 1A). We focused on comparing these groups as several studies have described biological differences between DNA methylation groups that provide important prognostic value. We utilized a computational pipeline that incorporates rMATS for predicting and quantifying AS events from RNA-seq data. AS events were classified into five types: cassette alternative exons (CA), mutually exclusive exons (MXE), alternative 3′ splice sites (A3SS), alternative 5′ splice sites (A5SS), and retained introns (RI), and for each AS event a “percent-spliced in” (PSI) score was calculated representing the ratio of isoforms including the event vs. others (FIG. 1B). Within the discovery cohort we identified 56 significant AS events (APSI>10%, FDR<0.05, p<0.05) between Immune-enriched and Hypermitotic meningiomas, 65 significant AS events between Immune-enriched and Merlin-intact meningiomas, and 107 significant AS events between Merlin-intact and Hypermitotic meningiomas, the majority of which were CA or MXE events across all DNA methylation groups (FIG. 1C). A total of 228 AS events were detected in these three comparisons, some of which were shared across multiple comparisons (FIG. 1D). A total of 184 unique AS events were used to cluster all tumors, including a cluster enriched for Hypermitotic meningiomas (FIGS. 1E, 1F). This subset of meningiomas were also enriched for high-risk subtypes from other DNA methylation classifiers, gene expression signatures, molecularly integrated grades, and CDKN2A deletions (data not shown). We also observed a non-significant trend towards higher proportions of meningiomas harboring TERT promoter (TERTp), BAP1, and SMARCB1 mutations (data not shown). Additionally, we compared recurrent vs. primary samples, identifying 13 significant AS events when considering all meningiomas regardless of DNA methylation group, 506 significant AS events between recurrent vs. primary Merlin-intact meningiomas, 104 significant AS events between recurrent vs. primary Immune-enriched meningiomas, and 33 significant AS events between recurrent vs. primary Hypermitotic meningiomas (data not shown).

Among the AS events significantly enriched in Hypermitotic meningiomas, we identified inclusion of a CA exon in NASP that generates a long NASP isoform (also known as testicular or tNASP) (FIGS. 1G, 1H). NASP is an essential gene in mammals that regulates cell cycle progression through histone maintenance and is dysregulated in multiple human tumor types. Depletion of tNASP can impair cancer cell proliferation in vitro. Interestingly, this event was not different across WHO grades but was enriched in recurrent meningioma samples (FIG. 1I). Importantly, stratifying meningioma samples based on NASP splicing demonstrated significantly worse outcomes in local freedom from recurrence (LFFR) (5-year LFFR [95% confidence interval]=54% [44-67%]NASP-CA high; 81% [74-87%]NASP-CA low; 71% [64-79%]NASP-CA intermediate) and overall survival (OS) for patients with high NASP-CA inclusion (5-year OS [95% confidence interval]=67% [58-78%]NASP-CA high; 88% [83-94%]NASP-CA low; 84% [78-89%]NASP-CA intermediate) (FIG. 1J). A second example of AS events distinguishing the three meningioma groups was an RI event in HNRNPM that was suppressed in higher WHO grade, the Hypermitotic DNA methylation group, and recurrent meningiomas (FIGS. 1K-1M). hnRNPM is a SF whose expression is frequently dysregulated in human tumors. Patients with low HNRNPM-RI inclusion had lower rates of LFFR (5-year LFFR [95% confidence interval]=81% [74-86%]HNRNPM-RI high; 51% [41%-62%]HNRNPM-RI low; 77% [71-83%]HNRNPM-RI intermediate) and OS (5-year OS [95% confidence interval]=83% [76-90%]HNRNPM-RI high; 70% [62-79%]HNRNPM-RI low; 88% [83-92%]HNRNPM-RI intermediate) (FIG. 1N). A third example of a gene impacted by AS was MFF which encodes a protein that regulates mitochondrial permeability and cell death pathways, and when inhibited decreases cancer cell viability. Multiple AS events were seen in MFF, including a CA event with decreased inclusion in WHO2, Hypermitotic, and recurrent meningiomas (FIGS. 10-1Q). Low inclusion of MFF-CA was associated with decreased rates of LFFR and OS (5-year LFFR [95% confidence interval]=86% [80-93%]MFF-CA high; 46% [38-57%]MFF-CA low; 79% [72-85%]MFF-CA intermediate) and OS (5-year OS [95% confidence interval]=88% [83-94%]MFF-CA high; 70% [62-78%]MFF-CA low; 86% [81-92%]MFF-CA intermediate) (FIG. 1R). We observed additional AS events in other transcripts whose splicing has previously been described to impact cancer phenotypes, including cancer cell metabolism (PFKM), cytoskeletal dynamics and angiogenesis (ADD3), and ribosome biogenesis and cell turnover (EIF4A2) (data not shown).

Given the prognostic value of individual AS events on predicting patient survival as described above, we next aimed to classify these tumors based on AS event PSI values de novo (data not shown). We utilized 1,000 AS events with the highest standard deviation across samples in the discovery cohort after filtering for read count (n_{supporting-reads}>10) and extremes of event inclusion or skipping (PSI_{Cohort-average}>90% or <10%) (data not shown). Hierarchical clustering using Euclidean distance resulted in 6 clusters with two (cluster 4 and 6) containing a disproportionate amount of Hypermitotic and Immune-enriched meningiomas compared to Merlin-intact meningiomas (data not shown). Compared to other groupings based on AS profiles, these groups demonstrated lower rates of LFFR but no significant impact on OS (data not shown). K-means clustering gave similar results with two clusters (cluster 2 and 3) showing higher rates of aggressive meningiomas and decreased LFFR without significant impact on OS (data not shown). Taken together, these data suggest that AS can be utilized as a powerful tool for assessing meningioma recurrence and patient prognosis and identifies potential aggressive subgroups with specific AS alterations.

### Alternative RNA Splicing can Reliably Predict Meningioma DNA Methylation Group

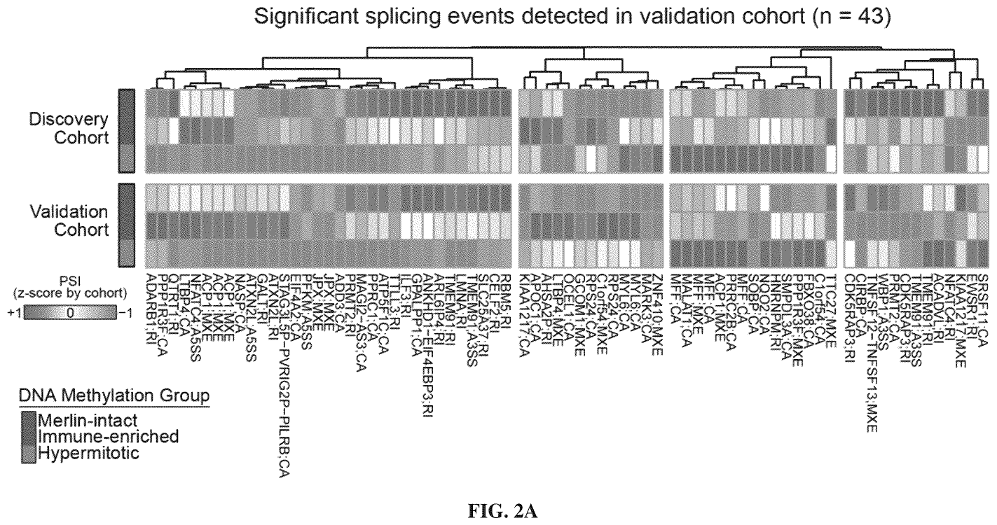

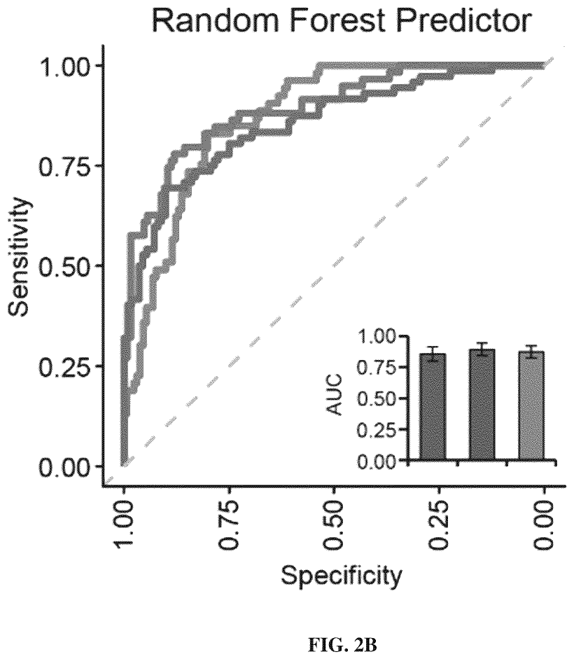

Since AS events differed between methylation groups, we next aimed to identify a set of AS events that could reliably predict DNA methylation signatures across patient cohorts. We therefore filtered the 184 significant AS events from the discovery cohort (FIG. 1E) for those that were also detected in the validation cohort, resulting in 74 AS events with very similar patterns of PSI differences across meningioma DNA methylation groups in both cohorts (FIG. 2A). We further filtered this set of events for those that could be identified across all tumor samples in both cohorts, resulting in 43 AS events that were utilized to train a random forest classifier with z-scaled PSI values from the discovery cohort, and used this model to predict DNA methylation groups of the validation cohort (FIG. 2B). This model had a high predictive value for each methylation group (AUC_{merlin-intact}=0.82 [0.75-0.88], AUC_{immune-enriched}=0.83 [0.77-0.89], AUC_{hypermitotic}=0.86 [0.81-0.91]) (FIG. 2B). Together, suggesting that AS profiles can be used to classify tumor DNA methylation group.

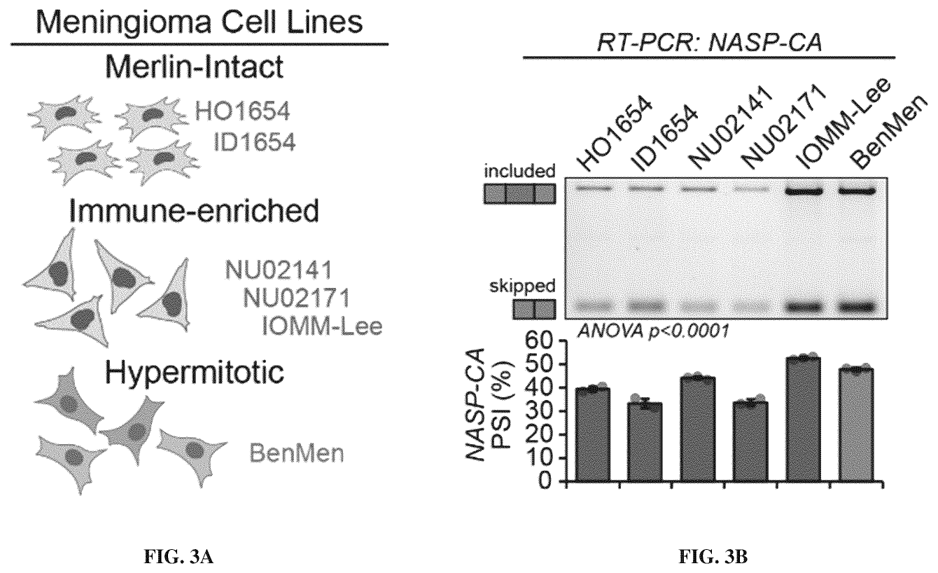

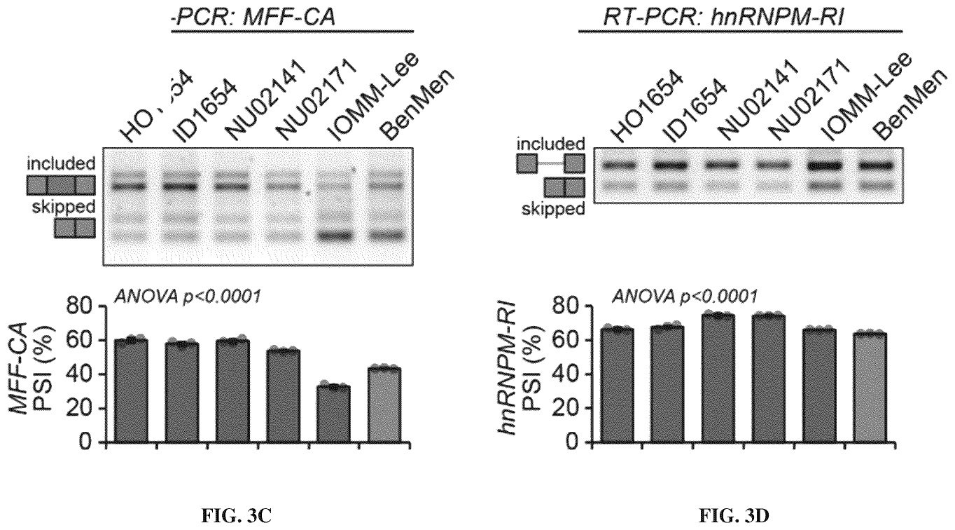

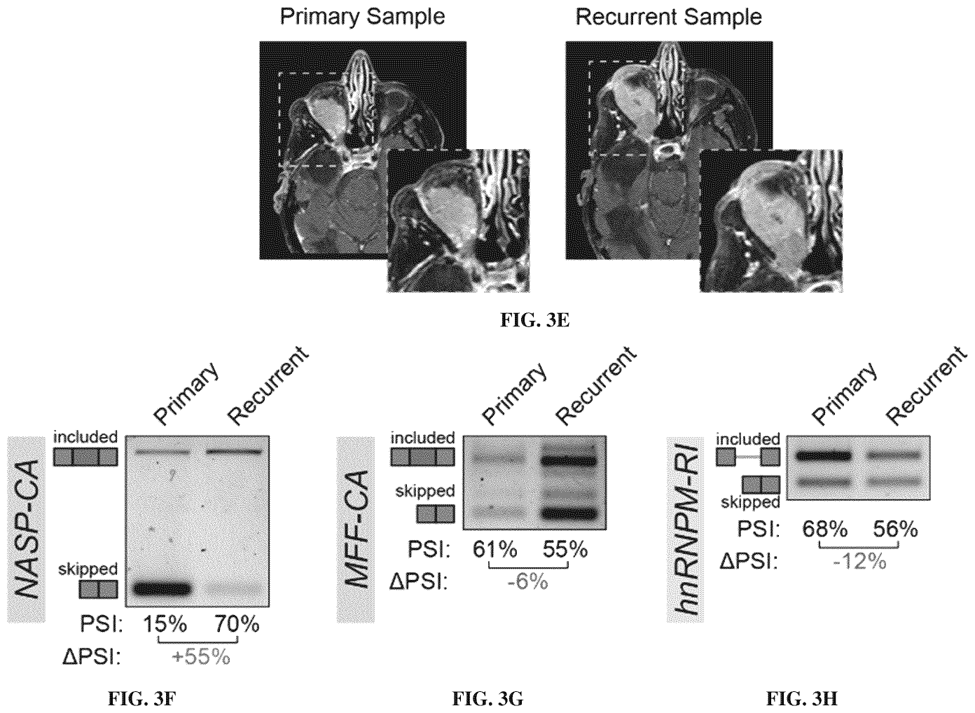

RT-PCR can Readily Detect Alternative RNA Splicing Changes in Human Meningioma Samples and Cell Lines Current methods to capture DNA methylation status from human tumors require sophisticated sequencing techniques with large upfront costs and time investment, which can restrict their use to large academic centers. Given that AS events can readily predict DNA methylation groups (FIGS. 2A-2B) we aimed to develop RT-PCR based assays to both validate the RNA-seq data and provide potential testing strategies. We focused on three events described above (NASP-CA, MFF-CA, HNRNPM-RI) given their biological significance and ability to stratify patient outcomes (FIGS. 1A-1R). We first validated these AS events in six meningioma cell lines with DNA-methylation patterns consistent with either Merlin-intact (HO1654, ID1654), Immune-enriched (NU02141, NU02171, IOMM-Lee), or Hypermitotic (BenMen) meningiomas (FIG. 3A) using RT-PCR with primers simultaneously detecting both RNA isoforms. While we observed some inherent variability between cell lines, BenMen cells had higher inclusion of NASP-CA, lower inclusion of MFF-CA and hnRNPM-RI then most other cell lines, consistent with primary Hypermitotic tumors (FIGS. 3B-3D). Notably, while classified as an Immune-enriched meningioma cell line, IOMM-Lee exhibited a highly proliferative growth rate and splicing patterns more consistent with Hypermitotic meningiomas (FIGS. 3B-3D).

We sought to further validate these AS events in a clinical scenario of tumor recurrence in a patient with a meningioma extending into the right orbit that underwent initial resection followed by recurrence (FIG. 3E). We observed in the recurrent vs. the primary tumor sample: increased exon inclusion of NASP-CA (ΔPSI=+55%), decreased exon inclusion of MFF-CA (ΔPSI=−6%), and decreased intron retention of HNRNPM-RI (ΔPSI=−12%) (FIGS. 3F-3H). Together, analysis of cell lines and an independent human sample supports the use of RT-PCR assays to detect splicing changes associated with aggressive meningioma behavior.

### RNA-Binding Proteins and Splicing Factors are Differentially Expressed Across Meningioma Groups

Since dysregulation of AS frequently occurs due to changes in RBP and SF expression, we aimed to assess differences in the splicing machinery across meningioma DNA methylation groups. We performed differential gene expression analysis on the discovery cohort, which readily clustered samples by DNA methylation group (data not shown). Overall, we observed 1,805 upregulated and 1,099 downregulated genes comparing Hypermitotic to Merlin-intact meningiomas; 1,347 upregulated and 1,887 downregulated genes comparing Hypermitotic to Immune-enriched meningiomas; and 2,788 upregulated and 1,347 downregulated genes comparing Immune-enriched to Merlin-intact meningiomas (upregulated: Log₂FC>1, p_{adj}<0.05; downregulated: Log₂FC<−1, p_{adj}<0.05) (data not shown). Consistent with previous studies, genes upregulated in Hypermitotic meningiomas were associated with mitosis, those in Immune-enriched meningiomas with immune cell activation and inflammatory responses, and those in Merlin-intact meningiomas with cellular differentiation and epidermal development (data not shown).

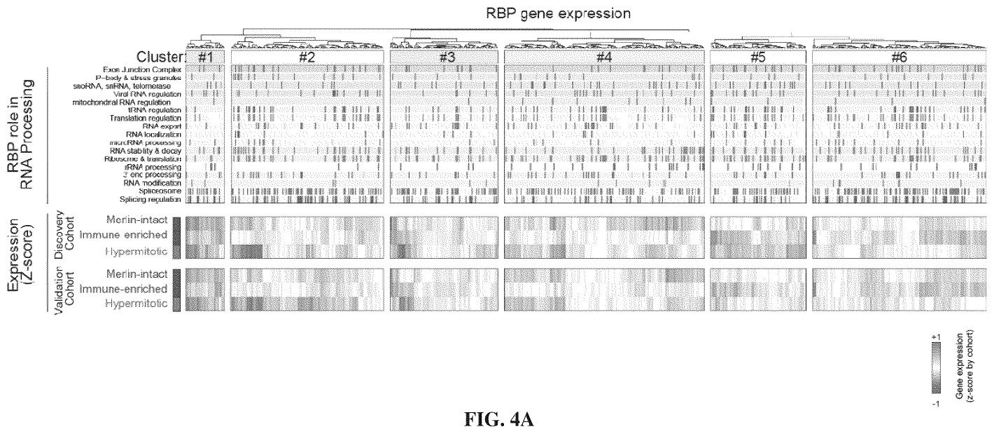

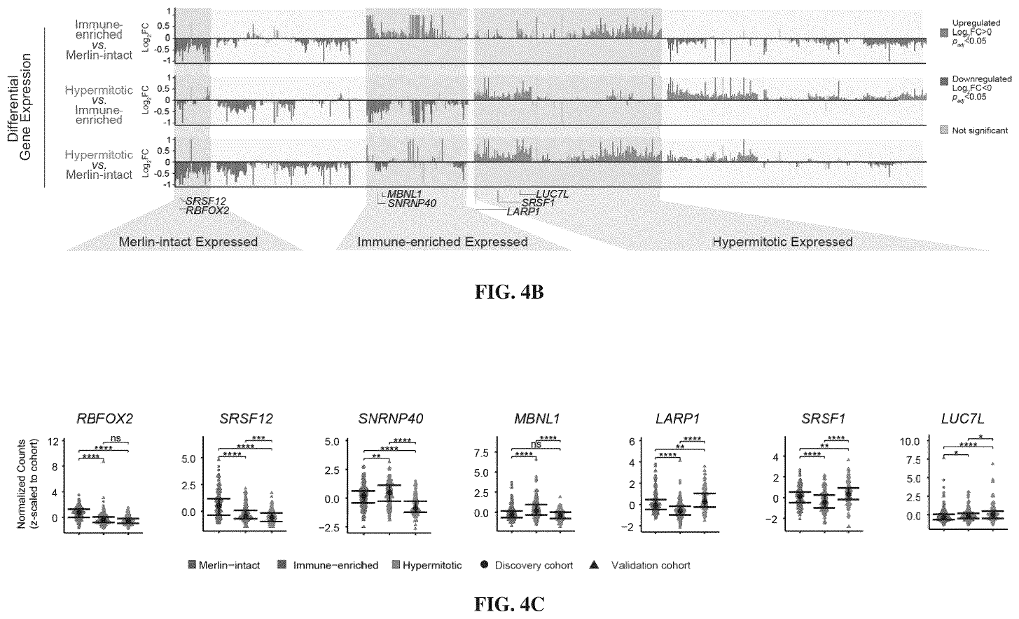

We next examined the expression of 770 annotated RBPs across DNA methylation groups using normalized count values z-scaled by cohort to control for any absolute differences between the cohorts. Tumors were classified into six clusters according to RBP expression: RBPs differentially expressed and preferentially upregulated in Merlin-intact (cluster #1: e.g., RBFOX2, SRSF12), Immune-enriched (cluster #3: e.g., SNRNP40, MBNL1), and Hypermitotic meningiomas (cluster #4: e.g., LARP1, SRSF1, LUC7L) (FIGS. 4A, 4B). The remaining clusters had more variable RBP expression. We further performed differential expression analysis of RBP genes annotated as directly associated with the spliceosome or as regulating AS, utilizing a lower FC cutoff (Log₂FC>101) as even small changes in SF-levels occurring in human tumors can cause profound effects on cellular phenotypes. We identified 76 upregulated (Log₂FC>0, p_{adj}<0.05) and 96 downregulated (Log₂FC<0, p_{adj}<0.05) SFs when comparing Immune-enriched vs. Merlin-intact, 103 upregulated and 69 downregulated SFs in Hypermitotic vs. Immune-enriched, and 83 upregulated and 87 downregulated SFs in Hypermitotic vs. Merlin-intact meningiomas (data not shown). Together these data demonstrate differential expression of RBPs and SFs across meningioma DNA-methylation groups and suggests the importance of these difference in driving underlying splicing changes.

### DDX39A and SRSF1 are Upregulated in Hypermitotic Meningiomas and Impact Proliferation

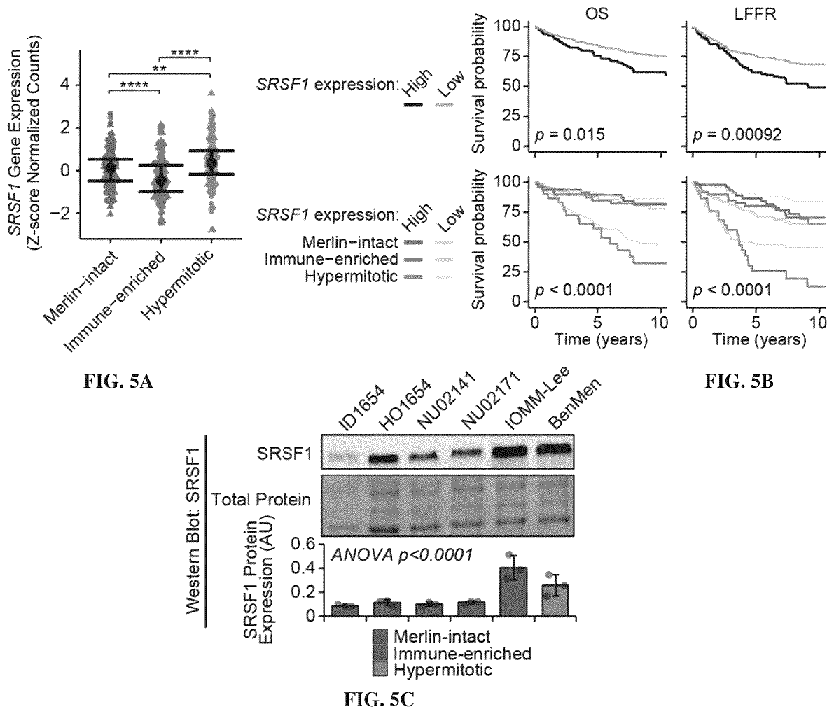

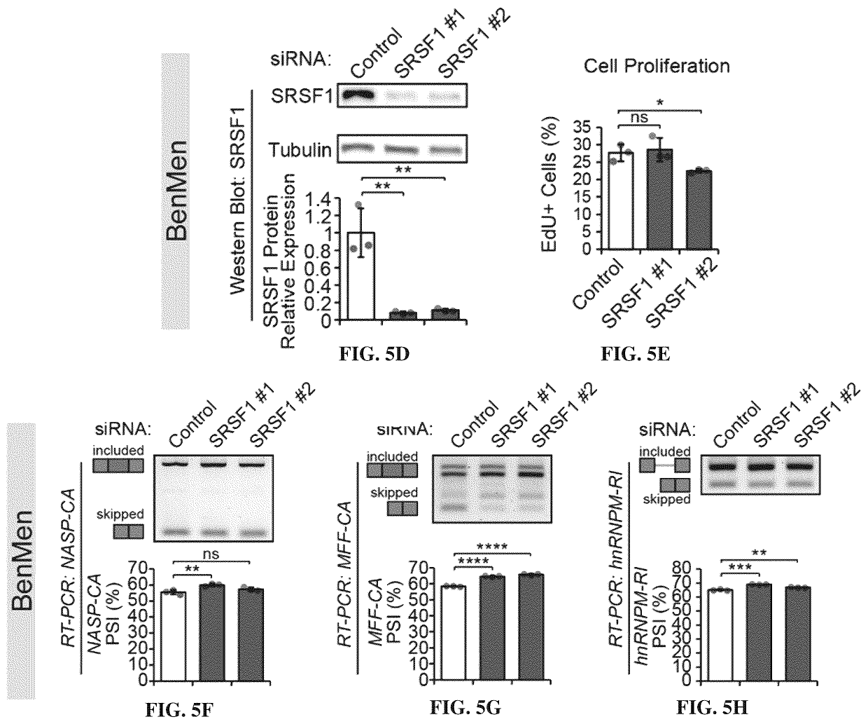

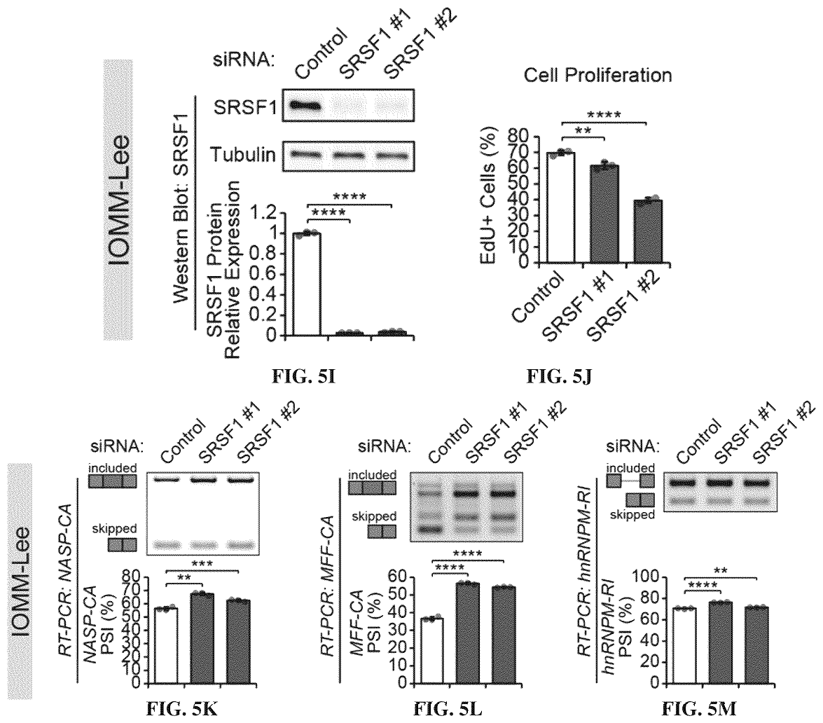

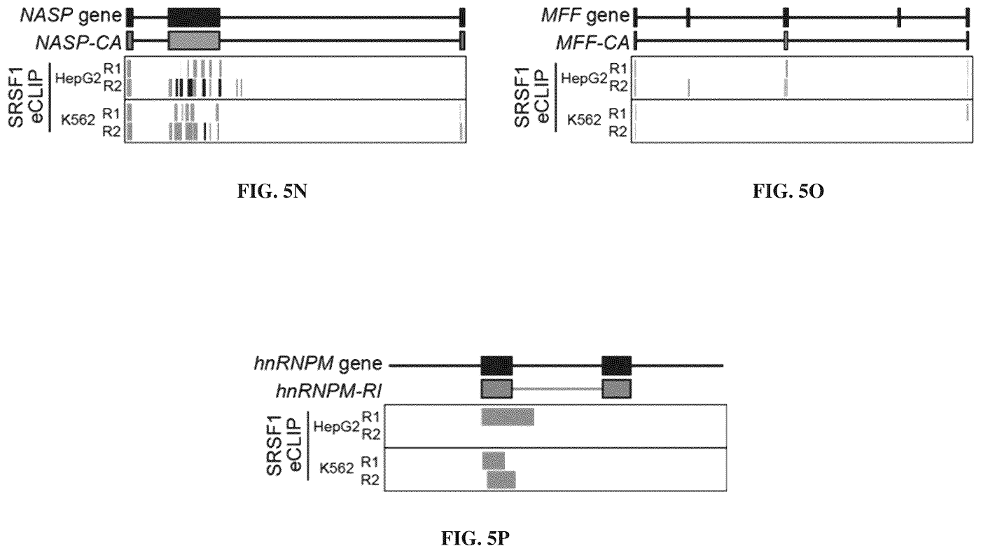

SFs can act as potent oncogenes when overexpressed in human cancers. We therefore focused on two RBPs, DDX39A and SRSF1, upregulated in Hypermitotic meningiomas (FIGS. 5A-5P) and previously implicated in other cancers. DDX39A is an RNA-helicase which functions in mRNA nuclear export and telomere maintenance, and has been associated with poor prognosis in pediatric neuroblastomas. We observed lower rates of OS and LFFR for patient samples with high DDX39A expression, both regardless of their DNA methylation status and when accounting for their underlying DNA methylation group (data not shown). BenMen and IOMM-Lee, two highly proliferative meningioma cell lines, exhibit higher levels of DDX39A protein compared to other cell lines (data not shown). Knockdown (KD) of DDX39A decreased proliferation in both IOMM-Lee and BenMen cells (data not shown) and had a reproducible but mild impact on AS of NASP-CA (ΔPSI=−5%) in BenMen cells (data not shown). However, DDX39A KD did not impact AS of MFF-CA and had inconsistent effects on HNRNPM-RI splicing across siRNAs (data not shown), suggesting that DDX39A might impacts meningioma cell phenotypes through alternative routes aside from its role in splicing. In comparison, SRSF1 is a well validated oncogenic SF in other human cancers where it promotes AS of pro-tumorigenic isoforms. We observed increased SRSF1 expression in Hypermitotic vs. Merlin-intact and Immune-enriched meningiomas (FIG. 5A). Meningioma patient samples with high SRSF1 expression, regardless of DNA methylation group, demonstrated lower OS and LFFR rates. Additionally, when incorporating DNA methylation information, high SRSF1 expression identified a group of Hypermitotic tumors that tended to have lower OS and LFFR (FIG. 5B). Finally, we observed higher SRSF1 protein levels in Hypermitotic BenMen cells and hyperproliferative Immune-enriched IOMM-Lee cells (FIG. 5C). Together, these data suggest high SRSF1 levels may contribute to meningioma aggressiveness.

To examine how SRSF1 expression influences meningioma cell phenotypes we utilized siRNAs to deplete SRSF1 in BenMen and IOMM-Lee cells. In both cell lines we achieved>90% SRSF1 protein KD and observed decreased cell proliferation and mis-splicing of target transcripts (FIGS. 5D-5M). Specifically, SRSF1 KD increased inclusion of MFF-CA and hnRNPM-RI (FIGS. 5G, 5L, 5H, 5M), suggesting that baseline SRSF1 expression promotes skipping of these events in Hypermitotic meningioma samples. Conversely, SRSF1 KD increased NASP-CA inclusion (FIGS. 5F, 5K), suggesting the increased inclusion of NASP-CA observed in Hypermitotic meningiomas is driven by other SFs, such as DDX39A or context-specific splicing in these samples. Changes in splicing of target transcripts following perturbation of SF-levels can be explained by both direct effects of the SF on the target and indirect effects caused by secondary changes in other SFs or binding site occupancy. To address this, we utilized publicly available enhanced cross-linking and immunoprecipitation (eCLIP-seq) ENCODE data from HepG2 hepatocellular carcinoma and K562 leukemia cell lines to examine SRSF1 binding around the NASP-CA, MFF-CA, and hnRNPM-RI AS events (FIGS. 5N-5P). SRSF1 eCLIP peaks were found, in at least one cell line, within the alternative exon and surrounding exonic sequences in NASP-CA and MFF-CA events (FIGS. 5N, 5O), as well as at the 5′ end of the hnRNPM-RI event (FIG. 5P). Together, these data suggest that SRSF1 plays a direct role in regulating of these AS events observed in human meningiomas.

### Therapeutic Targeting of Alternative Splicing Events in NASP and MFF

RNA splicing has been an attractive target for the design of targeted therapies, ranging from broad spectrum splicing inhibition to highly specific isoform-level targeting. Given the established role of NASP and MFF in tumorigenesis and their prognostic value in predicting meningioma patient outcomes (FIGS. 1A-1R), we aimed to develop splice-switching antisense oligonucleotide (ASO) to reverse NASP-CA and MFF-CA splicing changes observed in Hypermitotic meningiomas.

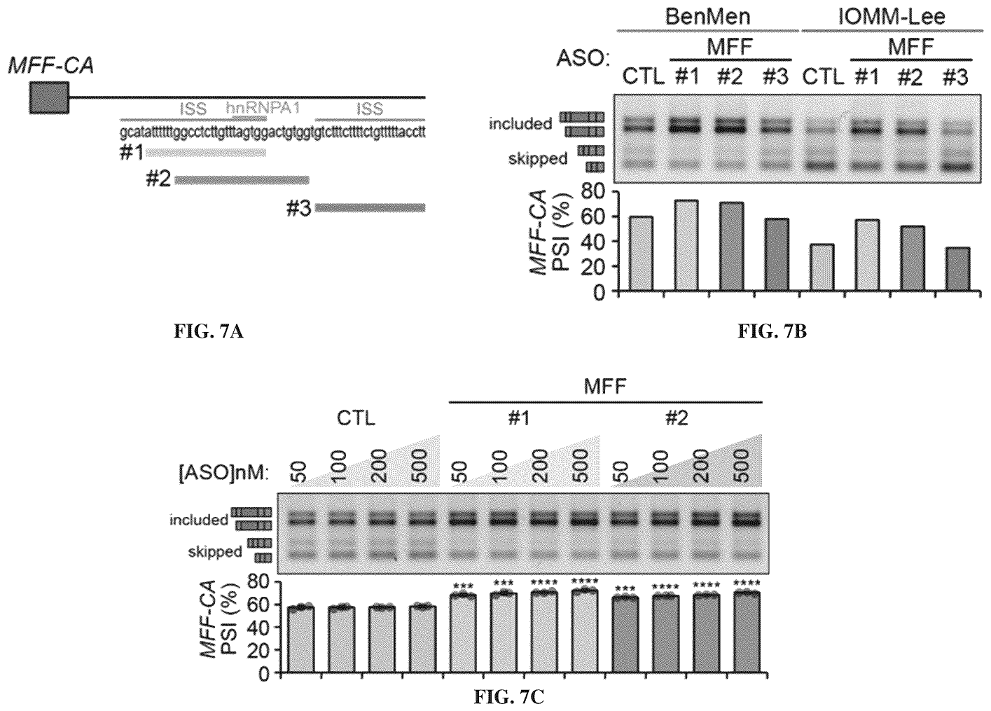

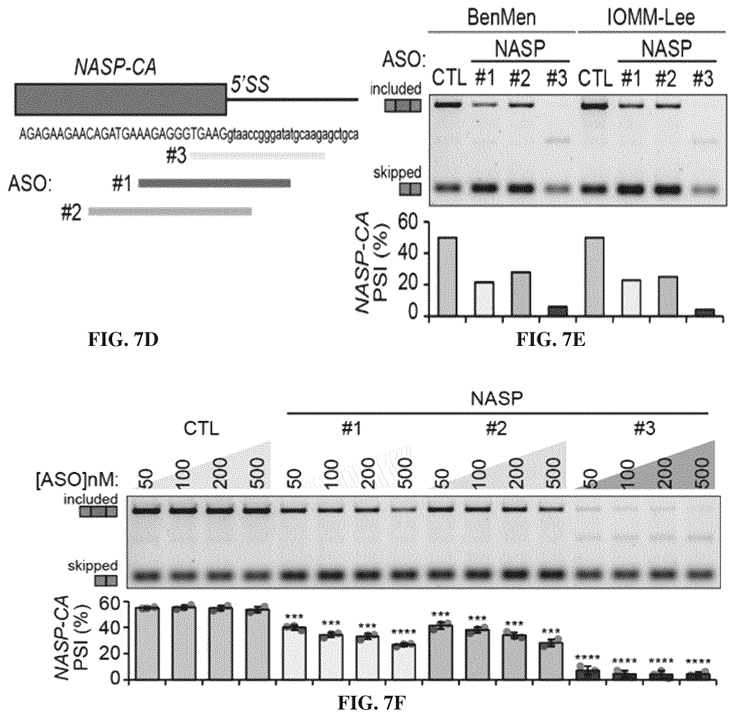

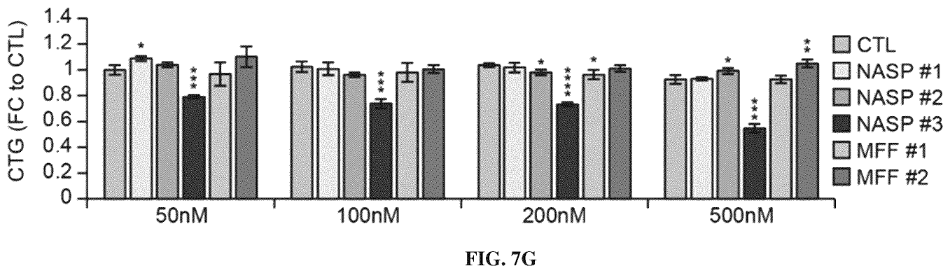

We designed three ASOs targeting an intronic splicing silencer and hnRNPA1 binding site upstream of MFF-CA to promote its inclusion (FIG. 7A) and reverse the low MFF-CA inclusion observed in Hypermitotic meningiomas. All three ASOs promoted MFF-CA inclusion, with ASO-MFF #1 and ASO-MFF #2 promoting strong dose-dependent effects (FIGS. 7B, 7C). While MFF-CA directed ASOs had very modest effects on cell viability in an initial screen in BenMen cells (FIG. 7G), we aimed to further explore the impact of MFF-CA splicing on meningioma cell phenotypes. Treatment of BenMen cells with ASO-MFF #1 or ASO-MFF #2 increased MFF-CA inclusion as well as relative levels of high-molecular weight (H-MW) to low-molecular weight (L-MW) MFF protein isoforms (data not shown). ASO-MFF treatment in BenMen cells induced variable results on cell proliferation with ASO-MFF #1 increasing and ASO-MFF #2 decreasing proliferation (data not shown). Additionally, we observed little to no impact of ASO-MFF #1 or ASO-MFF #2 on MFF localization in BenMen cells (data not shown). We observed similar results in IOMM-Lee cells (data not shown). Given the role of MFF and mTOR signaling in mitochondrial function, we co-treated BenMen and IOMM-Lee cells with ASO-MFF #1 or ASO-MFF #2 and the mTOR inhibitor everolimus (data not shown). Everolimus treatment reduced levels of phosphorylated S6 kinase and S6 ribosome and decreased proliferation of ASO-CTL treated BenMen and IOMM-Lee cells (data not shown). However, co-treatment of ASO-MFF and everolimus produced variable results on cell proliferation, with ASO-MFF #2 creating an additive reduction in proliferation of BenMen cells co-treated with everolimus (data not shown), while both ASO-MFF #1 and ASO-MFF #2 produced mild but significant increases in proliferation of IOMM-Lee cells co-treated with everolimus (data not shown). Together, these data suggest targeting MFF-CA with ASOs induces changes in MFF-CA splicing and MFF protein isoform expression. However, given its established role in mitochondrial metabolism and turnover, peroxisome biogenesis, and autophagy, the variable effects on cell proliferation may be related to cellular stress and metabolic pathways.

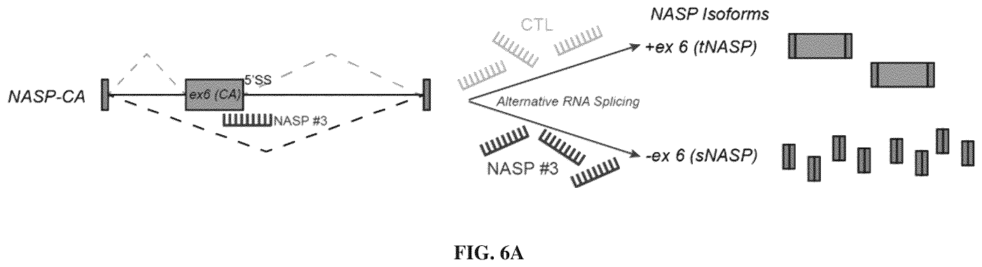

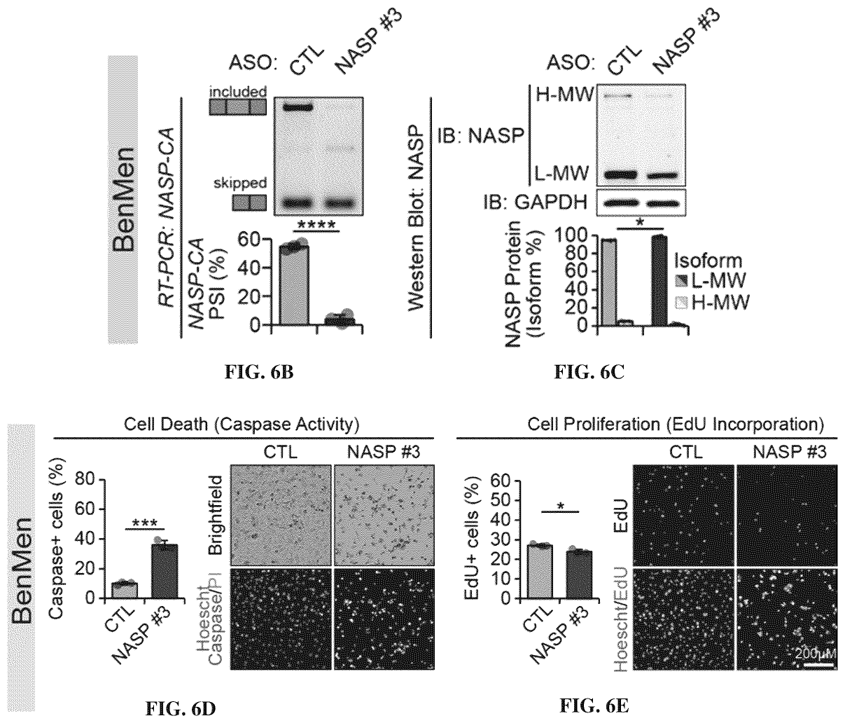

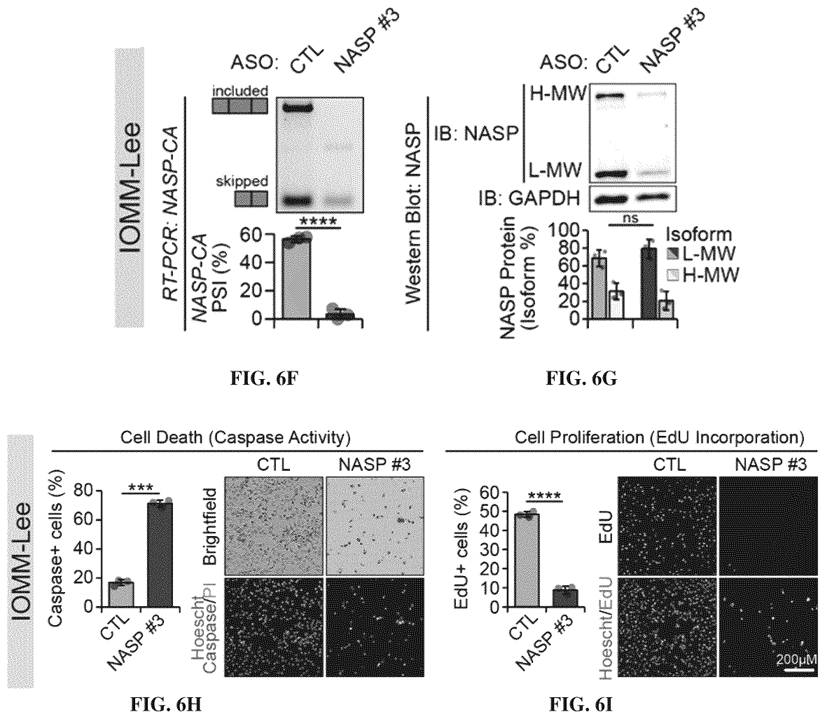

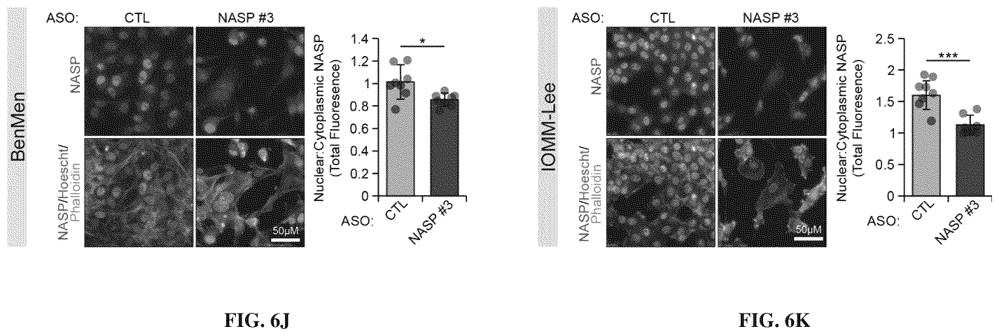

Similarly, we designed three splice-switching ASOs targeting the 5′ splice site (5′SS) of the NASP-CA event to block exon recognition and decrease NASP-CA inclusion, which is detected in Hypermitotic meningiomas (FIG. 6A). All ASOs significantly decreased NASP-CA inclusion in a dose dependent manner in IOMM-Lee and BenMen cells, with ASO-NASP #3 promoting almost complete exon skipping (data not shown). To further explore the cytotoxic effects of targeting NASP splicing, we treated BenMen and IOMM-Lee cells with control ASO or ASO-NASP #3 which led to a significant reduction in NASP-CA inclusion (FIGS. 6B, 6F), and decreased ratio of tNASP (H-MW) to sNASP (L-MW) protein isoforms (FIGS. 6C, 6G) in both cell lines. ASO-NASP #3 treatment significantly increased cell death (FC_{BenMen}=3.5, FC_{IOMM-Lee}=4.2) (FIGS. 6D, 6H), and decreased cell proliferation (FC_{BenMen}=0.89, FC_{IOMM-Lee}=0.18) (FIGS. 6E, 6I) compared to ASO-CTL, consistent with its impact on overall cell viability (data not shown). Cells treated with ASO-NASP #3 demonstrated redistribution of NASP protein from the nucleus to the cytoplasm (FIGS. 6J, 6K), consistent with previously reported nuclear predominance of tNASP vs. sNASP, likely do to differential inclusion of a 339 amino acid sequence that regulates its association with histone variants and its nuclear vs. cytoplasmic localization. Together, these data suggest that NASP-targeting ASOs induce cytotoxic effects through altering protein isoform abundance of tNASP/sNASP and its subcellular localization.

### Methods

### Human Cell Lines

HO1654, ID1654, NU02141, NU02171, IOMM-Lee, and BenMen cell lines are maintained in DMEM (Gibco) with 10% FBS, 1% penicillin streptomycin (Sigma), and 1× glutamax (Gibco). Cells are grown at 37° C. with 5% CO₂. Cells are routinely tested negative for mycoplasma using the MycoAlert™ Mycoplasma Detection Kit (Lonza), and early passages aliquots are used.

### Patient Samples

Meningioma tissue and MRI with brief clinical history were provided as deidentified samples from the University of Connecticut Health Center biobank (IRB #IE-08-310-1). MRI images shown are contrast-enhanced T1-weighted series. For RT-PCR analysis, meningioma tissue was lysed into RLT buffer (Qiagen) and continued through RNA extraction and PCR as below.

### RNA Extraction and Reverse Transcription

Cells are lysed using RLT buffer (Qiagen) supplemented with 1% β-Mercaptoethanol. RNA is purified using an RNAeasy kit (Qiagen) with DNAse I. 250-500 ng of RNA is reverse transcribed using Superscript III reverse transcriptase (Invitrogen).

### Semi-Quantitative PCR for Splicing Detection

20 ng cDNA is amplified with Phusion hot start II DNA polymerase (Thermo Fisher) and primers. PCR products are separated in 1-2% agarose gel stained with SYBR Safe (Invitrogen) and imaged using ChemiDoc MP Imaging System (Bio-rad). PCR bands are quantified using ImageLab 6.0 (Bio-rad) and the percent spliced-in (PSI) ratio of each transcript is calculated as the exon-included band intensity divided by the intensity of included and skipped isoform bands. ΔPSI is calculated as PSI_{case}−PSI_{control}.

### Cell Line Transfections

Cell lines are reverse transfected with siRNAs (Ambion Silencer Select siRNA) or uniformly modified 2′-methoxyethyl (2′MOE) ASOs with phosphorothioate backbones (IDT) using Lipofectamine RNAiMAX (Invitrogen). siRNAs and ASOs are diluted to final concentration of 10 nM and 50-500 nM, respectively, in 100 uL Optimum media (Gibco), supplemented with 1.5 μL of lipofectamine RNAiMAX. Following incubation at room temperature, 2.5×10⁵ cells/mL of resuspended cells in 500 μL media are added to the siRNA or ASO mix. 125 ul or 500 μL of the mix are platted into 96- or 24-well plates for phenotyping or RNA and protein extraction. For everolimus co-treatment, cells were transfected with ASOs as above and plated in everolimus (Thermo) containing media to final concentration of 10 nM.

### Western Blot Analysis

Cells are harvested in 2 mM EDTA in PBS and lysed in Laemmli buffer (50 mM Tris-HCl pH 6.2, 5% β-mercaptoethanol, 10% glycerol, 3% SDS). Protein lysates are ran on 8-16% gradient gels (Biorad), transferred onto nitrocellulose membranes (Millipore) and blocked with 5% milk in Tween 20-TBST (50 mM Tris pH 7.5, 150 mM NaCl, 0.05% Tween 20). Blots are incubated with primary and secondary antibodies, and imaged with a ChemiDoc MP Imaging System (Bio-rad). Protein expression is quantified using ImageLab 6.0 software (Bio-rad), normalized to loading control and expressed as fold change (FC) to controls.

### Phenotypic Assays

ASO or siRNA-treated cells are seeded into 96 well imaging plates (Perkin Elmer) at 2.5×10⁴ cells per well. For caspase activation: 48 h after transfection cells are incubated for 1 h with 5 μM Cell Event Caspase-3/7 detection reagent (Invitrogen) and 5 ng/mL Hoechst (Life Technologies). For cell proliferation: 48 h after transfection, cells are labelled with 10 μM EdU for 6 h, fixed in 4% paraformaldehyde, and permeabilized with 0.5% tritonX-100. EdU is detected using the Click-iT cell proliferation kit (Thermo Fisher) with alexa-647 azide, and counterstained with Hoechst (5 ng/mL). For all assays, nine fields of view per replicate are imaged with a 10× objective on an Opera Phenix high-content imaging system (Perkin Elmer). Caspase+ or EdU+ cells and total Hoechst+ nuclei are counted using the Columbus analysis software (Perkin Elmer) and presented as the percentage of Caspase+ or EdU+ cells.

### Immunofluorescence

48 h following transfection cells were washed with PBS, fixed with 4% paraformaldehyde, washed with IF buffer (7.6 g/L NaCl, 1.896 g/L Na₂HPO₄, 0.414 g/L NaH₂PO₄, 0.5 g/L NaN₃, 1 g/L BSA, 0.2% Triton X-100, 0.05% Tween-20, pH 7.4), permeabilized with 0.5% TritonX-100, and blocked with 10% goat serum (Sigma). Cells were counterstained with Hoechst and phalloidin-647 (Thermo) and imaged with a 20× objective using an Opera Phenix high-content imaging system (Perkin Elmer).

### Human Meningioma Cohorts

RNA-seq data from human meningioma samples was previously published (GSE183653, GSE212666). For each analysis, meningioma samples were split between a discovery (GSE212666 n=302, 150 bp paired-ended reads) and validation (GSE183653, n=184, 50 bp single-ended reads) cohort as described.

### Differential Splicing and Survival Analysis in Human Meningioma Samples

Differential splicing analysis is carried out using an in-house computational pipeline that incorporates rMATS for event level splicing quantification (v2.0 github.com/TheJacksonLaboratory/splicing-pipelines-nf). To stratify patients based on splicing, PSI values for individual splicing events were extracted and samples were categorized as “high inclusion” (z-score>0.5), “low inclusion” (z-score<−0.5), or “other” (−0.5<z-score<0.5). Survival analysis was performed using Survival and Survminer R packages.

### Differential Gene Expression Analysis in Human Meningioma Samples

Differential gene expression is performed using DESeq2⁸⁰ in R with gene count matrices from STAR mapped fastq files filtered for read counts>10, by comparing DNA-methylation groups (hypermitotic vs. merlin-intact, hypermitotic vs. immune-enriched, immune-enriched vs. merlin-intact). Significant differential expression is assessed using p_{adjusted}-value<0.05 (Benjamini-Hochberg). To compare splicing-factor expression, normalized gene counts were z-scored for each cohort and plotted with median gene expression compared using a Wilcoxon test.
Visualization of eCLIP and ChIP-Seq Data from ENCODE/ENCORE
ENCODE data for SRSF1 eCLIP-seq from HepG2 (ENCSR989VIY) and K562 (ENCSR432XUP) was visualized in the UCSC genome browser as peak call outputs from ENCODE analysis^{48,81}.

### Gene Ontology Analysis Using Enrichr

Gene lists from differential expression analysis were analyzed with Enrichr (https://maayanlab.cloud/Enrichr/). Results for GO Biological Processes 2023 were plotted using R.

### Graphs and Figures

Plots were generated in R (v3.6.3) or excel (Microsoft) and then formatted using Illustrator (Adobe). Figures were generated using Illustrator (Adobe) in compliance with the Nature Publishing Group policy concerning image integrity. Figures were supplemented with images from BioRender.

### Quantification and Statistical Analysis

Plots include mean±stdev or median±interquartile range, as well as individual replicates/samples were applicable. For RT-PCR, western blot, and immunofluorescence data is presented as the mean±stdev and significant differences to a control are assessed using a two-tailed unpaired t-test. For plots generated in R, statistics are done using the ggpubr package.

SSO NASP Target Sequences
NASP SSO Target #1 AAGAGGGTGAAGGTAACCGGGATA (SEQ ID NO: 5)
NASP SSO Target #2 CAGATGAAAGAGGGTGAAGGTAAC (SEQ ID NO: 6)
NASP SSO Target #3 TGAAGGTAACCGGGATATGCAAGA (SEQ ID NO: 2)
SSO NASP Sequences
NASP SSO #1 TATCCCGGTTACCTTCACCCTCTT (SEQ ID NO: 7)
NASP SSO #2 GTTACCTTCACCCTCTTTCATCTG (SEQ ID NO: 8)
NASP SSO #3 TCTTGCATATCCCGGTTACCTTCA (SEQ ID NO: 4)
Chemically Modified SSO NASP Sequences (2′-O-methoxyethly-RNA with uniformly
modified phosphorothioate backbones and 5′-methylcytosines)*
Key: i2MOErX = internal 2′-O-methoxyethly X
52MOErX = 5′ 2′-O-methoxyethyl X
32MOErX = 3′ 2′-O-methoxyethyl X
/*/ = phosphorothioate bond
X = adenine, cytosine, guanine, or thymine
NASP SSO #1
/52MOErT/*/i2MOErA/*/i2MOErT/*/i2MOErC/*/i2MOErC/*/i2MOErC/*/i2MOErG/*/
i2MOErG/*/i2MOErT/*/i2MOErT/*/i2MOErA/*/i2MOErC/*/i2MOErC/*/i2MOErT/*/i2MOErT
/*/i2MOErC/*/i2MOErA/*/i2MOErC/*/i2MOErC/*/i2MOErC/*/i2MOErT/*/i2MOErC/*/
i2MOErT/*/32MOErT/ (SEQ ID NO: 9)
NASP SSO #2
/52MOErG/*/i2MOErT/*/i2MOErT/*/i2MOErA/*/i2MOErC/*/i2MOErC/*/i2MOErT/*/
i2MOErT/*/i2MOErC/*/i2MOErA/*/i2MOErC/*/i2MOErC/*/i2MOErC/*/i2MOErT/*/
i2MOErC/*/i2MOErT/*/i2MOErT/*/i2MOErT/*/i2MOErC/*/i2MOErA/*/i2MOErT/*/i2MOErC/*/
i2MOErT/*/32MOErG/ (SEQ ID NO: 10)
NASP SSO #3
/52MOErT/*/i2MOErC/*/i2MOErT/*/i2MOErT/*/i2MOErG/*/i2MOErC/*/i2MOErA/*/
i2MOErT/*/i2MOErA/*/i2MOErT/*/i2MOErC/*/i2MOErC/*/i2MOErC/*/i2MOErG/*/
i2MOErG/*/i2MOErT/*/i2MOErT/*/i2MOErA/*/i2MOErC/*/i2MOErC/*/i2MOErT/*/i2MOErT/*/
i2MOErC/*/32MOErA/ (SEQ ID NO: 11)
SSO MFF Target Sequences
MFF SSO Target #1 ATTTTTTGGCCTCTTGTTTAGTGG (SEQ ID NO: 12)
MFF SSO Target #2 GGCCTCTTGTTTAGTGGACTGTGG (SEQ ID NO: 13)
MFF SSO Target #3 GTCTTTCTTTTCTGTTTTTACCTT (SEQ ID NO: 14)
SSO MFF Sequences
MFF SSO #1 CCACTAAACAAGAGGCCAAAAAAT (SEQ ID NO: 15)
MFF SSO #2 CCACAGTCCACTAAACAAGAGGCC (SEQ ID NO: 16)
MFF SSO #3 AAGGTAAAAACAGAAAAGAAAGAC (SEQ ID NO: 17)
Chemically Modified MFF NASP Sequences (2′-O-methoxyethly-RNA with uniformly
modified phosphorothioate backbones and 5′-methylcytosines)
MFF SSO #1
/52MOErC/*/i2MOErC/*/i2MOErA/*/i2MOErC/*/i2MOErT/*/i2MOErA/*/i2MOErA/*/
i2MOErA/*/i2MOErC/*/i2MOErA/*/i2MOErA/*/i2MOErG/*/i2MOErA/*/i2MOErG/*/
i2MOErG/*/i2MOErC/*/i2MOErC/*/i2MOErA/*/i2MOErA/*/i2MOErA/*/i2MOErA/*/i2MOErA/
*/i2MOErA/*/32MOErT/ (SEQ ID NO: 18)
MFF SSO #2
/52MOErC/*/i2MOErC/*/i2MOErA/*/i2MOErC/*/i2MOErA/*/i2MOErG/*/i2MOErT/*/
i2MOErC/*/i2MOErC/*/i2MOErA/*/i2MOErC/*/i2MOErT/*/i2MOErA/*/i2MOErA/*/
i2MOErA/*/i2MOErC/*/i2MOErA/*/i2MOErA/*/i2MOErG/*/i2MOErA/*/i2MOErG/*/
i2MOErG/*/i2MOErC/*/32MOErC/ (SEQ ID NO: 19)
MFF SSO #3
/52MOErA/*/i2MOErA/*/i2MOErG/*/i2MOErG/*/i2MOErT/*/i2MOErA/*/i2MOErA/*/
i2MOErA/*/i2MOErA/*/i2MOErA/*/i2MOErC/*/i2MOErA/*/i2MOErG/*/i2MOErA/*/
i2MOErA/*/i2MOErA/*/i2MOErA/*/i2MOErG/*/i2MOErA/*/i2MOErA/*/i2MOErA/*/
i2MOErG/*/i2MOErA/*/32MOErC/ (SEQ ID NO: 20)

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. An engineered splice-switching antisense oligonucleotide that binds to the nucleotide sequence of SEQ ID NO: 1.

2. The engineered splice-switching antisense oligonucleotide of claim 1 that binds to the nucleotide sequence of SEQ ID NO: 2.

3. An engineered splice-switching antisense oligonucleotide comprising the nucleotide sequence of SEQ ID NO: 3.

4. The engineered splice-switching antisense oligonucleotide of claim 1 comprising the nucleotide sequence of SEQ ID NO: 4 or a nucleotide sequence having at least 90% identity to the nucleotide sequence of SEQ ID NO: 4.

5. The engineered splice-switching antisense oligonucleotide of claim 4 comprising a nucleotide sequence having at least 95% identity to the nucleotide sequence of SEQ ID NO: 4.

6. The engineered splice-switching antisense oligonucleotide of claim 5 comprising the nucleotide sequence of SEQ ID NO: 4.

7. The engineered splice-switching antisense oligonucleotide of claim 1 comprising a chemical modification.

8. The engineered splice-switching antisense oligonucleotide of claim 7, wherein the chemical modification is selected from backbone modifications, sugar modifications, and base modifications.

9. The engineered splice-switching antisense oligonucleotide of claim 8, wherein the chemical modification is a backbone modification.

10. The engineered splice-switching antisense oligonucleotide of claim 9, wherein the backbone modification is a phosphorothioate (PS) modification.

11. The engineered splice-switching antisense oligonucleotide of claim 8, wherein the chemical modification is a sugar modification.

12. The engineered splice-switching antisense oligonucleotide of claim 11, wherein the sugar modification is 2′-O-Methyl (2′-OMe) or 2′-O-Methoxyethyl (2′-MOE).

13. A composition comprising the engineered splice-switching antisense oligonucleotide of claim 1 and an excipient.

14. A composition comprising the engineered splice-switching antisense oligonucleotide claim 1 associated with a delivery vehicle.

15. The composition of claim 14, wherein the delivery vehicle is selected from lipid nanoparticles, liposomes, polymeric nanoparticles, gold nanoparticles, peptide-based delivery systems, aptamer-based delivery systems, exosomes, viral vectors, and molecules capable of crossing the blood-brain barrier.

16. The composition of claim 13, wherein the concentration of the engineered splice-switching antisense oligonucleotide in the composition is about 2 mg/ml to about 200 mg/ml.

17. A method comprising administering the engineered splice-switching antisense oligonucleotide of claim 1 to a subject, wherein the subject has a meningioma.

18. The method of claim 17, wherein the engineered splice-switching antisense oligonucleotide is administered via intrathecal injection, intracranial injection, or intravenous infusion.

19. The method of claim 17, wherein the meningioma is classified as Hypermitotic meningioma.

20. (canceled)

21. A method comprising administering the engineered splice-switching antisense oligonucleotide of claim 1 to a cell, optionally a meningioma cell, in an amount effective to modify splicing of NASP transcript.

22. (canceled)
